(19) United States
(12) Patent Application Publication
(10) Pub. No.: US 2009/0062183 A1
(43) Pub. Date: Mar. 5, 2009

(54) Modulation of synaptogenesis

(75) Inventors: Ben A. Barres, Palo Alto, CA (US); Karen Sue Christopherson, San Mateo, CA (US); Erik M. Ullian, San Mateo, CA (US)

Correspondence Address: MORRISON & FOERSTER LLP, 755 PAGE MILL RD, PALO ALTO, CA, 94304-1018, US

(73) Assignee: The Board of Trustees of the Leland Stanford Junior University

(21) Appl. No.: 12/291,133

(22) Filed: Nov. 5, 2008

Related U.S. Application Data
(63) Continuation of application No. 11/176,450, filed on Jul. 6, 2005, now abandoned.
(60) Provisional application No. 60/586,960, filed on Jul. 8, 2004.

(51) Int. Cl. A61K 38/16; G01N 33/567

(52) U.S. Cl. 514/8; 435/7.21

(57) ABSTRACT

Soluble proteins, e.g. thrombospondins, can trigger synapse formation. Such proteins are synthesized in vitro and in vivo by astrocytes, which therefore have a role in synaptogenesis. These thrombospondins are only expressed in the normal brain exactly during the period of developmental synaptogenesis, being off in embryonic brain and adult brain but on at high levels in postnatal brain. Methods are provided for protecting or treating an individual suffering from adverse effects of deficits in synaptogenesis, or from undesirably active synaptogenesis. These findings have broad implications for a variety of clinical conditions, including traumatic brain injury, epilepsy, and other conditions where synapses fail to form or form inappropriately. Synaptogenesis is enhanced by contacting neurons with agents that are specific agonists or antagonists of thrombospondins. Conversely, synaptogenesis is inhibited by contacting neurons with inhibitors or antagonists of thrombospondins.

### BACKGROUND OF THE INVENTION

Synapses are specialized cell adhesions that are the fundamental functional units of the nervous system, and they are generated during development with amazing precision and fidelity. During synaptogenesis, synapses form, mature, and stabilize and are also eliminated by a process that requires intimate communication between pre- and postsynaptic partners. In addition, there may be environmental determinants that help to control the timing, location, and number of synapses.

Synapses occur between neuron and neuron and, in the periphery, between neuron and effector cell, e.g. muscle. Functional contact between two neurons may occur between axon and cell body, axon and dendrite, cell body and cell body, or dendrite and dendrite. It is this functional contact that allows neurotransmission. Many neurologic and psychiatric diseases are caused by pathologic overactivity or underactivity of neurotransmission; and many drugs can modify neurotransmission, for examples hallucinogens and antipsychotic drugs.

During recent years, a great deal of effort has been made by investigators to characterize the function of synaptic proteins, which include synaptotagmin, syntexin, synaptophysin, synaptobrevin, and the synapsins. These proteins are involved in specific aspects of synaptic function, e.g. synaptic vesicle recycling or docking, and in the organization of axonogenesis, differentiation of presynaptic terminals, and in the formation and maintenance of synaptic connections.

Only by establishing synaptic connections can nerve cells organize into networks and acquire information processing capability such as learning and memory. Synapses are progressively reduced in number during normal aging, and are severely disrupted during neurodegenerative diseases. Therefore, finding molecules capable of creating and/or maintaining synaptic connections is an important step in the treatment of neurodegenerative diseases.

The modulation of synapse formation is of great interest for the treatment of a variety of nervous system disorders. To date, no soluble molecule has been identified that is sufficient to induce or increase the number of CNS synapses.

### SUMMARY OF THE INVENTION

Methods are provided for the modulation of synaptogenesis with soluble factors. It has been found that thrombospondin is sufficient to increase synapse formation on neurons. Thrombospondin, or agonists and mimetics thereof, are administered to enhance synaptogenesis. Thrombospondin inhibitors or antagonists are administered to decrease synaptogenesis.

In one embodiment of the invention, methods are provided for screening candidate agents for an ability to modulate synapse formation. In one embodiment of the invention the neurons are neurons in the central nervous system. In another embodiment, the neurons are peripheral nervous system neurons.

Methods are provided for protecting or treating an individual suffering from adverse effects of deficits in synaptogenesis, or from undesirably active synaptogenesis. These findings have broad implications for a variety of clinical conditions, including traumatic brain injury, epilepsy, and other conditions where synapses fail to form or form inappropriately. Synaptogenesis is enhanced by contacting neurons with agents that are specific agonists or antagonists of thrombospondins. Conversely, synaptogenesis is inhibited by contacting neurons with inhibitors or antagonists of thrombospondins.

### BRIEF DESCRIPTION OF THE DRAWINGS

FIG. 1. Cholesterol and apolipoprotein E are not sufficient to increase synapse number. (A) Immunostaining of RGCs for colocalization of presynaptic synaptotagmin (red) and postsynaptic PSD-95 (green) shows few synaptic puncta in the absence of astrocytes (control), but many in the presence of astrocyte conditioned medium (ACM) or a feeding layer of astrocytes (astros), indicating that astrocytes secrete a synapse-promoting activity that is also active in ACM. (B) Astrocyte feeding layer (astros) increases frequency of spontaneous mEPSCs above control while ACM does not. (C) Synapse-promoting activity in ACM is over 100 KD. ACM was concentrated with molecular weight cut-off (MWCO) filters of 5, 50, and 100 KD. The number of puncta from ACM prepared with a 100 KD MWCO filter is similar to the number of puncta produced by astrocyte feeding layer, indicating that the astrocyte-derived synapse-promoting activity is over 100 KD. (D) Immunodepletion of cholesterol-containing ApoE complexes from ACM with an ApoE-specific antibody. (E, F) ApoE-depleted ACM retains full synapse-promoting activity indicating that cholesterol bound to ApoE is not the synapse-promoting activity in ACM. Asterisks in all panels correspond to p<0.05 compared to control.

FIG. 2. TSP1 mimics synapse-promoting activity of ACM. (A) Immunostaining for colocalization of presynaptic synaptotagmin (red) and postsynaptic PSD-95 (green) shows few RGC synaptic puncta in the absence of astrocytes (control), but many in the presence of thrombospondin 1 (TSP1), indicating that TSP1 alone is sufficient to increase synaptic puncta on neurons. Cholesterol induces no increase in puncta. (B) Quantification of the effects of ACM, TSP1, and ACM+TSP1 on synaptic puncta. ACM and TSP1 significantly increase the number of synaptic puncta over control. ACM+TSP1 increases synaptic puncta to the same extent as either ACM or TSP1 alone, indicating that the effect of ACM is not additive with the effect of TSP1. (C) Cholesterol does not increase the number of synaptic puncta in neurons. (D) Measurement of the number of spontaneous mEPSCs recorded in neurons cultured with cholesterol or an astrocyte feeding layer (astros) indicates a significant increase in spontaneous event frequency in neurons cultured with cholesterol compared to control, but a much bigger increase in frequency in neurons cultured with an astrocyte feeding layer. Inset show spontaneous activity examples in neurons cultured with cholesterol or astrocyte feeding layer. Astrocyte feeding layers cause a coordinated bursting of massive synaptic events not seen in the presence of cholesterol. (E) Cumulative amplitude distribution of spontaneous mEPSCs measured in neurons cultured with cholesterol (dashed line) or astrocyte feeding layer (solid line) indicates that the amplitude population of mEPSCs is much smaller in neurons cultured with cholesterol compared to an astrocyte feeding layer. Asterisks in all panels correspond to p<0.05 compared to control.

FIG. 3. TSP1 induces ultrastructurally normal synapses. (A) Electron micrographs (EM) of synapses in the presence of ACM, TSP1 or astrocyte feeding layer (astros). In all cases ultrastructurally normal synapses are seen. (B) Quantification of total number of vesicles (black bars) and number of docked vesicles (gray bars) per synapse per section indicates no difference between synapses formed in the presence of ACM, TSP1, or astros indicating that all three promote formation of normal and indistinguishable ultrastructural synapses. (C) Quantification of the number of synapses per cell per section measured by EM shows a significant increase in the number of synapses on neurons cultured with ACM, TSP1, or astros compared to control. Asterisks correspond to p<0.05 compared to control

FIG. 4. TSP2 is necessary for the increase in synapse number induced by ACM. (A) Immunostaining for colocalization of presynaptic synaptotagmin (red) and postsynaptic PSD-95 (green) shows few RGC synaptic puncta in the absence of astrocytes (control), but many in the presence of recombinant TSP2. (B) Quantification of the increase in synaptic puncta with rTSP2 indicates that rTSP2 is sufficient to increase the number of structural synapses. Asterisks correspond to p<0.01 compared to control. (C) Immunodepletion with a TSP2-specific antibody depletes TSP2 (TSP2 beads) from ACM (TSP2 depl ACM). (D) Quantification indicates that TSP2-depleted ACM reduces synapse-promoting activity to control. Asterisks correspond to p<0.05 compared to control. (E) Mock-depleted ACM retains full synapse-promoting activity (left panel and inset; synaptotagmin, red, PSD-95, green) while TSP2-depleted ACM is depleted of synapse-promoting activity (right panel and inset). TSP2-depleted ACM promotes an increase in the number of pre- and post-synaptic labeling on neurons, but the puncta are no longer colocalized.

FIG. 5. TSP1-induced synapses are presynaptically active but postsynaptically silent. (A) Measurement of spontaneous mEPSCs shows that neither ACM nor TSP1 increase event frequency above control levels, in contrast to a feeding layer of astrocytes (astros). (B) Rocs treated with ACM, TSP1, and astrocyte feeding layer (astros) all have significantly more presynaptic uptake of an anti-synaptotagmin luminal domain antibody than neurons cultured alone (control), indicating that ACM- and TSP1-induced synapses are presynaptically active. (C) Whole-cell L-glutamate responses indicate that ACM and TSP1 do not increase postsynaptic responses to glutamate above control levels, in contrast to astrocyte feeding layers (astros). Inset depicts the postsynaptic glutamate response in an RGC grown with an astrocyte feeding layer, indicating that it is mediated by non-NMDA receptors. (D) Measurement of cumulative amplitude distributions reveals that neither ACM nor TSP1 increase mEPSC amplitudes above control, in contrast to astrocyte feeding layers. This indicates that few functional glutamate receptors are present at synaptic sites. These results indicates that TSP1 and ACM do not increase postsynaptic glutamate receptor expression or function, and is consistent with TSP1 and ACM inducing postsynaptically silent, but presynaptically functional synapses. Asterisks in all panels correspond to p<0.05 compared to control.

FIG. 6. TSP1/2 immunoreactivity is localized to astrocyte processes at many synapses throughout the developing brain. (A) Confocal images of immunolabelled rat postnatal day 8 (p8) brain sections reveals TSP1/2 throughout the cortex (left panel) as well as presynaptic puncta labeled with synaptotagmin (SYN; middle panel). TSP1/2 is located at synaptic sites as indicated by the double labeling for TSP1/2 and SYN in the merged image (right panel). (B) Confocal images of immunolabelled p8 superior colliculus (SC) reveal TSP1/2 throughout neuropil (left panel) as well as SYN puncta (middle panel). Merged images shows overlap of SYN and TSP1/2 in SC (right panel). (C) Immunolabelling of cortex with TSP1/2 (left panel) and the fine glial process marker ezrin (middle panel) reveals extensive punctate labeling. Merged images reveals overlap of ezrin and TSP1/2 (right panel) indicating that TSP1/2 is located to fine astrocyte processes, many of which surround synapses. Arrows in all panels indicate labeled puncta. (D) Western blot analysis of p5 rat cortical lysates shows that both TSP1 (left panel) and TSP2 (right panel) proteins are present in postnatal cortex and down regulated in adult cortex.

FIG. 7. Quantification of synapse number in TSP1/2 double-null brain. (A) Confocal sections of cortical fields immunostained for synaptic marker SV2 in WT P21 brain (left panel) and TSP1/2 double-null P21 brain (right panel) a. (B) Quantification of synapse number in matched cortical fields from P8 WT and TSP1/2 double-null brains. A significant reduction in synapse number in TSP1/2 double-null brains was found (p=0.0046). (C) Quantification of synapse number in matched cortical fields from P21 WT and TSP1/2 double-null brains. A significant reduction in synapse number in TSP1/2 double-null brains was found (p=0.0169). (D) MAP2 immunostaining in WT P21 brain (top panel) and matchedTSPl/2 double-nullP21 brain (bottom panel). (E) Quantification of dendritic area shows no difference in dendritic fields (p>0.05).

FIG. 8. TSP does not increase outgrowth in RGC cultures. (A) Example of a dye-filled RGC in culture for 10 days in the presence of TSP1. (B) Quantification of total process length per cell for dye-filled neurons showed no increase process length in RGCs cultured with TSP. The mean process length per cell was lower in TSP-treated cultures compared to control. (p=0.0043).

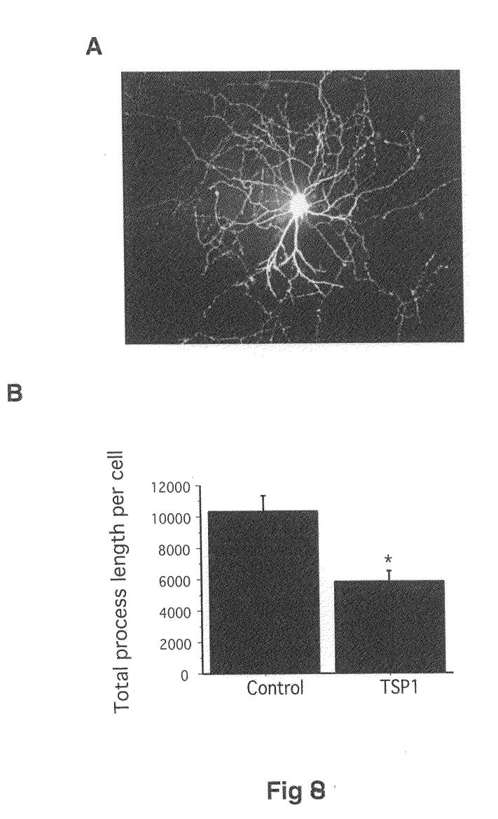

FIG. 9. Cholesterol increases quantal content of autaptic RGCs. (A) Example of an autaptic RGC grown in the presence of an astrocyte feeding layer and immunostained for presynaptic synaptotagmin (red) and postsynaptic PSD-95 (green). (B) Example of evoked EPSC recorded from an autaptic RGC cultured in the presence of cholesterol. (C) Measurement of the quantal content of autaptic RGCs cultured in the presence of unconcentrated astrocyte conditioned medium (1×ACM) or 10-fold concentrated ACM (10×ACM), or cholesterol. Cholesterol increased the quantal content of the neurons to the same level as 10×ACM. Asterisks correspond to p<0.05 compared to control.

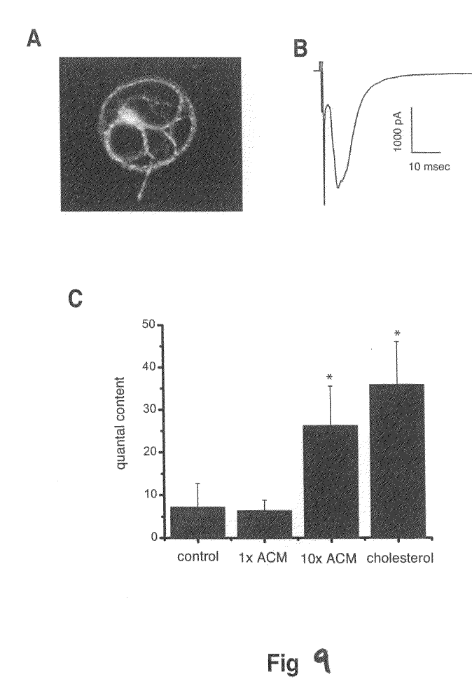

FIG. 10. TSP-2 does not increase synaptic activity in purified RGCs in vitro. (A) Example of spontaneous EPSCs measured by patch clamp recording in purified RGCs cultured under a feeding layer of astrocytes. The average frequency of spontaneous events is 400±100 events per minute in the presence of TTX. (B) Example of patch clamp recording from neurons treated with rTSP2. Despite the presence of structural synapses, no spontaneous events were recorded in neurons treated under these conditions (n=5).

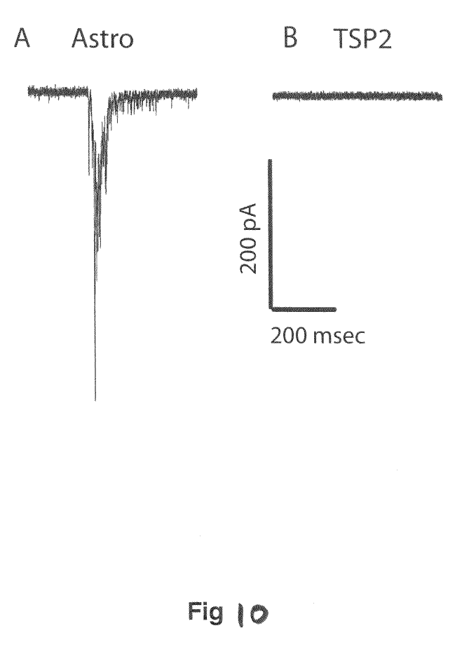

FIG. 11 is a bar graph. RGCs were cultured together with astrocyte inserts, or treated with 5 μg/ml TSP1, TSP4 and TSP5, or with culture media conditioned by cos7 cells overexpressing murine TSP3 for 6 days. TSP 3, 4 and 5 each induced an increase in synapse number similar to astrocytes or TSP1. Each bar indicates the number of co-localized puncta.

### DETAILED DESCRIPTION OF THE INVENTION

Methods are provided for protecting or treating an individual suffering from adverse effects of deficits in synaptogenesis, or from undesirably active synaptogenesis. These findings have broad implications for a variety of clinical conditions, including traumatic brain injury, epilepsy, and other conditions where synapses fail to form or form inappropriately. Synaptogenesis is enhanced by contacting neurons with agents that are specific agonists or antagonists of thrombospondins. Conversely, synaptogenesis is inhibited by contacting neurons with inhibitors or antagonists of thrombospondins.

It is demonstrated herein that soluble proteins, e.g. thrombospondins, can trigger synapse formation. Such proteins are synthesized in vitro and in vivo by astrocytes, which therefore have a role in synaptogenesis. These thrombospondins are only expressed in the normal brain exactly during the period of developmental synaptogenesis, being off in embryonic brain and adult brain but on at high levels in postnatal brain.

Delivery of an exogenous thrombospondin or an agonist thereof induces new synapses in normal CNS, after CNS injury to promote repair, at neuromuscular junctions, e.g. at the junctions of spinal motor neurons and muscles. The ability to restore synaptogenesis in an adult has important implications for enhancing memory in normal brain; for treatment of Alzheimer's disease (a disease where synapses are lost), as well as promoting new synaptogenesis in repair and regeneration of injured CNS after stroke or spinal cord injury; enhancement of neuromuscular junctions in muscular dystrophy; and the like. Delivery of an exogenous thrombospondin or an agonist thereof also find use in combination with administration of neural progenitors, or increases in neurogenesis, in order to promote functional connections between the nascent neurons and other neurons and effector cells.

Thrombospondin antagonists are useful in treating diseases of excess, unwanted synapses. The adult brain may upregulate thrombospondin after injury in “reactive astrocytes”, which form glial scars. Glial scars are associated with epileptic loci, and may induce the unwanted excess synaptogenesis that underlies epilepsy. Similarly there are unwanted extra synapses that underlie the long-lived drug craving of addiction.

### DEFINITIONS

Synaptogenesis. Synaptogenesis, as used herein, refers to the process by which pre- and/or post-synapses form on a neuron. Enhancing synaptogenesis results in an increased number of synapses, while inhibiting synaptogenesis results in a decrease in the number of synapses, or a lack of increase where an increase would otherwise occur. By “augmentation” or “modulation” of synaptogenesis as used herein, it is meant that the number of synapses formed is either enhanced or suppressed as required in the specific situation. As used herein, the term “modulator of synaptogenesis” refers to an agent that is able to alter synapse formation. Modulators include, but are not limited to, both “activators” and “inhibitors”. An “activator” or “agonist” is a substance that enhances synaptogenesis. Conversely, an “inhibitor” or “antagonist” decreases the number of synapses. The reduction may be complete or partial. As used herein, modulators encompass thrombospondin antagonists and agonists.

Agonists and antagonists may include proteins, nucleic acids, carbohydrates, antibodies, or any other molecules that decrease the effect of a protein. The term “analog” is used herein to refer to a molecule that structurally resembles a molecule of interest but which has been modified in a targeted and controlled manner, by replacing a specific substituent of the reference molecule with an alternate substituent. Compared to the starting molecule, an analog may exhibit the same, similar, or improved utility. Synthesis and screening of analogs, to identify variants of known compounds having improved traits (such as higher potency at a specific receptor type, or higher selectivity at a targeted receptor type and lower activity levels at other receptor types) is an approach that is well known in pharmaceutical chemistry.

Synapses are asymmetric communication junctions formed between two neurons, or, at the neuromuscular junction (NMJ) between a neuron and a muscle cell. Chemical synapses enable cell-to-cell communication via secretion of neurotransmitters, whereas in electrical synapses signals are transmitted through gap junctions, specialized intercellular channels that permit ionic current flow. In addition to ions, other molecules that modulate synaptic function (such as ATP and second messenger molecules) can diffuse through gap junctional pores. At the mature NMJ, pre- and postsynaptic membranes are separated by a synaptic cleft containing extracellular proteins that form the basal lamina. Synaptic vesicles are clustered at the presynaptic release site, transmitter receptors are clustered in junctional folds at the postsynaptic membrane, and glial processes surround the nerve terminal.

Synaptogenesis is a dynamic process. During development, more synapses are established than ultimately will be retained. Therefore, the elimination of excess synaptic inputs is a critical step in synaptic circuit maturation. Synapse elimination is a competitive process that involves interactions between pre- and postsynaptic partners. In the CNS, as with the NMJ, a developmental, activity-dependent remodeling of synaptic circuits takes place by a process that may involve the selective stabilization of coactive inputs and the elimination of inputs with uncorrelated activity. The anatomical refinement of synaptic circuits occurs at the level of individual axons and dendrites by a dynamic process that involves rapid elimination of synapses. As axons branch and remodel, synapses form and dismantle with synapse elimination occurring rapidly.

A number of cell adhesion molecules and tyrosine kinase receptor ligands have been implicated in modulating synaptogenesis. Integrins, cadherins, and neuroligins, are cell adhesion molecules that may play a role in synapse formation. The ephrins and their receptors, the Eph tyrosine kinases, participate in the activity-independent topographic organization of brain circuits and may also participate in synapse formation and maturation. Neurotrophins have also been implicated in aspects of synapse development and function.

Thrombospondin. As used herein, the term “thrombospondin” may refer to any one of the family of proteins which includes thrombospondins I, II, III, IV, and cartilage oligomeric matrix protein. Reference may also be made to one or more of the specific thrombospondins. Thrombospondin is a homotrimeric glycoprotein with disulfide-linked subunits of MW 180,000. It contains binding sites for thrombin, fibrinogen, heparin, fibronectin, plasminogen, plasminogen activator, collagen, laminin, etc. It functions in many cell adhesion and migration events, including platelet aggregation.

Thrombospondin I (THBS1) has the Genbank accession number X04665. It is a multimodular secreted protein that associates with the extracellular matrix and possesses a variety of biologic functions, including a potent angiogenic activity. Other thrombospondin genes include thrombospondins II (THBS2; 188061), III (THBS3; 188062), and IV (THBS4; 600715).

Human thrombospondin 2 (THBS2) has the Genbank accession number L12350. It is very similar in sequence to THBS1.

Human thrombospondin 3 (THBS3) has the Genbank accession number L38969. The protein is clearly homologous to THBS1 and THBS2 in its COOH-terminal domains but substantially different in its NH2-terminal region, suggesting functional properties for THBS3 that are unique, but also related to those of THBS1 and THBS2. The 956-amino acid predicted protein is highly acidic, especially in the third quarter of the sequence which corresponds to 7 type III calcium binding repeats. Four type II EGF-like repeats are also present.

The human THBS4 gene, Genbank accession number Z19585, contains an RGD (arg-gly-asp) cell-binding sequence in the third type 3 repeat. It is a pentameric protein that binds to heparin and calcium.

Cartilage oligomeric matrix protein, Genbank accession L32137, is a 524-kD protein that is expressed at high levels in the territorial matrix of chondrocytes. The sequences indicate that it is a member of the thrombospondin gene family.

For use in the subject methods, any of the native thrombospondin forms, modifications thereof, or a combination of forms may be used. Peptides of interest include fragments of at least about 12 contiguous amino acids, more usually at least about 20 contiguous amino acids, and may comprise 30 or more amino acids, up to the complete polypeptide.

The sequence of the thrombospondin polypeptide may be altered in various ways known in the art to generate targeted changes in sequence. The polypeptide will usually be substantially similar to the sequences provided herein, i.e. will differ by at least one amino acid, and may differ by at least two but not more than about ten amino acids. The sequence changes may be substitutions, insertions or deletions. Scanning mutations that systematically introduce alanine, or other residues, may be used to determine key amino acids. Conservative amino acid substitutions typically include substitutions within the following groups: (glycine, alanine); (valine, isoleucine, leucine); (aspartic acid, glutamic acid); (asparagine, glutamine); (serine, threonine); (lysine, arginine); or (phenylalanine, tyrosine).

Modifications of interest that do not alter primary sequence include chemical derivatization of polypeptides, e.g., acetylation, or carboxylation. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine.

Also included in the subject invention are polypeptides that have been modified using ordinary molecular biological techniques and synthetic chemistry so as to improve their resistance to proteolytic degradation or to optimize solubility properties or to render them more suitable as a therapeutic agent. For examples, the backbone of the peptide may be cyclized to enhance stability (see Friedler et al. (2000) J. Biol. Chem. 275:23783-23789). Analogs of such polypeptides include those containing residues other than naturally occurring L-amino acids, e.g. D-amino acids or non-naturally occurring synthetic amino acids.

The subject peptides may be prepared by in vitro synthesis, using conventional methods as known in the art. Various commercial synthetic apparatuses are available, for example, automated synthesizers by Applied Biosystems, Inc., Foster City, Calif., Beckman, etc. By using synthesizers, naturally occurring amino acids may be substituted with unnatural amino acids. The particular sequence and the manner of preparation will be determined by convenience, economics, purity required, and the like.

If desired, various groups may be introduced into the peptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

### Conditions of Interest

By “neurological” or “cognitive” function as used herein, it is meant that the increase of synapses in the brain enhances the patient's ability to think, function, etc. In conditions where there is axon loss and regrowth, there may be recovery of motor and sensory abilities. As used herein, the term “subject” encompasses mammals and non-mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. The term does not denote a particular age or gender.

Among the conditions of interest for the present methods of enhancing synaptogenesis are senescence, stroke, spinal cord injury, Alzheimer's disease (a disease where synapses are lost), as well as promoting new synaptogenesis in repair and regeneration of injured CNS after stroke or spinal cord injury. Such conditions benefit from administration of thrombospondin or thrombospondin agonists, which increase, or enhance, the development of synapses. In some instances, where there has been neuronal loss, it may be desirable to enhance neurogenesis as well, e.g. through administration of agents or regimens that increase neurogenesis, transplantation of neuronal progenitors, etc.

The term “stroke” broadly refers to the development of neurological deficits associated with impaired blood flow to the brain regardless of cause. Potential causes include, but are not limited to, thrombosis, hemorrhage and embolism. Current methods for diagnosing stroke include symptom evaluation, medical history, chest X-ray, ECG (electrical heart activity), EEG (brain nerve cell activity), CAT scan to assess brain damage and MRI to obtain internal body visuals. Thrombus, embolus, and systemic hypotension are among the most common causes of cerebral ischemic episodes. Other injuries may be caused by hypertension, hypertensive cerebral vascular disease, rupture of an aneurysm, an angioma, blood dyscrasias, cardiac failure, cardic arrest, cardiogenic shock, septic shock, head trauma, spinal cord trauma, seizure, bleeding from a tumor, or other blood loss.

By “ischemic episode” is meant any circumstance that results in a deficient supply of blood to a tissue. When the ischemia is associated with a stroke, it can be either global or focal ischemia, as defined below. The term “ischemic stroke” refers more specifically to a type of stroke that is of limited extent and caused due to blockage of blood flow. Cerebral ischemic episodes result from a deficiency in the blood supply to the brain. The spinal cord, which is also a part of the central nervous system, is equally susceptible to ischemia resulting from diminished blood flow.

By “focal ischemia,” as used herein in reference to the central nervous system, is meant the condition that results from the blockage of a single artery that supplies blood to the brain or spinal cord, resulting in damage to the cells in the territory supplied by that artery.

By “global ischemia,” as used herein in reference to the central nervous system, is meant the condition that results from a general diminution of blood flow to the entire brain, forebrain, or spinal cord, which causes the death of neurons in selectively vulnerable regions throughout these tissues. The pathology in each of these cases is quite different, as are the clinical correlates. Models of focal ischemia apply to patients with focal cerebral infarction, while models of global ischemia are analogous to cardiac arrest, and other causes of systemic hypotension.

Stroke can be modeled in animals, such as the rat (for a review see Duverger et al. (1988) J Cereb Blood Flow Metab 8(4):449-61), by occluding certain cerebral arteries that prevent blood from flowing into particular regions of the brain, then releasing the occlusion and permitting blood to flow back into that region of the brain (reperfusion). These focal ischemia models are in contrast to global ischemia models where blood flow to the entire brain is blocked for a period of time prior to reperfusion. Certain regions of the brain are particularly sensitive to this type of ischemic insult. The precise region of the brain that is directly affected is dictated by the location of the blockage and duration of ischemia prior to reperfusion. One model for focal cerebral ischemia uses middle cerebral artery occlusion (MCAO) in rats. Studies in normotensive rats can produce a standardized and repeatable infarction. MCAO in the rat mimics the increase in plasma catecholamines, electrocardiographic changes, sympathetic nerve discharge, and myocytolysis seen in the human patient population.

The methods of the invention are also useful for treatment of injuries to the central nervous system that are caused by mechanical forces, such as a blow to the head or spine, and which, in the absence of treatment, result in neuronal death, or severing of axons. Trauma can involve a tissue insult such as an abrasion, incision, contusion, puncture, compression, etc., such as can arise from traumatic contact of a foreign object with any locus of or appurtenant to the head, neck, or vertebral column. Other forms of traumatic injury can arise from constriction or compression of CNS tissue by an inappropriate accumulation of fluid (for example, a blockade or dysfunction of normal cerebrospinal fluid or vitreous humor fluid production, turnover, or volume regulation, or a subdural or intracranial hematoma or edema). Similarly, traumatic constriction or compression can arise from the presence of a mass of abnormal tissue, such as a metastatic or primary tumor.

Senescence refers to the effects or the characteristics of increasing age, particularly with respect to the diminished ability of somatic tissues to regenerate in response to damage, disease, and normal use. Alternatively, aging may be defined in terms of general physiological characteristics. The rate of aging is very species specific, where a human may be aged at about 50 years; and a rodent at about 2 years. In general terms, a natural progressive decline in body systems starts in early adulthood, but it becomes most evident several decades later.

One arbitrary way to define old age more precisely in humans is to say that it begins at conventional retirement age, around about 60, around about 65 years of age. Another definition sets parameters for aging coincident with the loss of reproductive ability, which is around about age 45, more usually around about 50 in humans, but will, however, vary with the individual. Loss of synaptic function may be found in aged individuals.

Among the aged, Alzheimer's disease is a serious condition. Alzheimer's disease is a progressive, inexorable loss of cognitive function associated with an excessive number of senile plaques in the cerebral cortex and subcortical gray matter, which also contains β-amyloid and neurofibrillary tangles consisting of tau protein. The common form affects persons >60 yr old, and its incidence increases as age advances. It accounts for more than 65% of the dementias in the elderly.

The cause of Alzheimer's disease is not known. The disease runs in families in about 15 to 20% of cases. The remaining, so-called sporadic cases have some genetic determinants. The disease has an autosomal dominant genetic pattern in most early-onset and some late-onset cases but a variable late-life penetrance. Environmental factors are the focus of active investigation.

In the course of the disease, neurons are lost within the cerebral cortex, hippocampus, and subcortical structures (including selective cell loss in the nucleus basalis of Meynert), locus caeruleus, and nucleus raphae dorsalis. Cerebral glucose use and perfusion is reduced in some areas of the brain (parietal lobe and temporal cortices in early-stage disease, prefrontal cortex in late-stage disease). Neuritic or senile plaques (composed of neurites, astrocytes, and glial cells around an amyloid core) and neurofibrillary tangles (composed of paired helical filaments) play a role in the pathogenesis of Alzheimer's disease. Senile plaques and neurofibrillary tangles occur with normal aging, but they are much more prevalent in persons with Alzheimer's disease.

The essential features of dementia are impairment of short-term memory and long-term memory, abstract thinking, and judgment; other disturbances of higher cortical function; and personality change. Progression of cognitive impairment confirms the diagnosis, and patients with Alzheimer's disease do not improve.

The methods of the invention find also find use in combination with cell or tissue transplantation to the central nervous system, where such grafts include neural progenitors such as those found in fetal tissues, neural stem cells, embryonic stem cells or other cells and tissues contemplated for neural repair or augmentation. Neural stem/progenitor cells have been described in the art, and their use in a variety of therapeutic protocols has been widely discussed. For example, inter alia, U.S. Pat. No. 6,638,501, Bjornson et al.; U.S. Pat. No. 6,541,255, Snyder et al.; U.S. Pat. No. 6,498,018, Carpenter; U.S. Patent Application 20020012903, Goldman et al.; Palmer et al. (2001) Nature 411(6833):42-3; Palmer et al. (1997) Mol Cell Neurosci. 8(6):389404; Svendsen et al. (1997) Exp. Neurol. 148(1):135-46 and Shihabuddin (1999) Mol Med. Today. 5(11):474-80; each herein specifically incorporated by reference.

Neural stem and progenitor cells can participate in aspects of normal development, including migration along well-established migratory pathways to disseminated CNS regions, differentiation into multiple developmentally- and regionally-appropriate cell types in response to microenvironmental cues, and non-disruptive, non-tumorigenic interspersion with host progenitors and their progeny. Human NSCs are capable of expressing foreign transgenes in vivo in these disseminated locations. A such, these cells find use in the treatment of a variety of conditions, including traumatic injury to the spinal cord, brain, and peripheral nervous system; treatment of degenerative disorders including Alzheimer's disease, Huntington's disease, Parkinson's disease; affective disorders including major depression; stroke; and the like. By synaptogenesis enhancers, the functional connections of the neurons are enhances, providing for an improved clinical outcome.

Among the conditions of interest for the present methods of decreasing synaptogenesis are epilepsy, and drug addition. Such conditions benefit from administration of thrombospondin or thrombospondin antagonists, which decrease, or inhibit, the development of synapses.

Epilepsy is a recurrent, paroxysmal disorder of cerebral function characterized by sudden, brief attacks of altered consciousness, motor activity, sensory phenomena, or inappropriate behavior caused by excessive discharge of cerebral neurons. Manifestations, depend on the type of seizure, which may be classified as partial or generalized. In partial seizures, the excess neuronal discharge is contained within one region of the cerebral cortex. In generalized seizures, the discharge bilaterally and diffusely involves the entire cortex. Sometimes a focal lesion of one part of a hemisphere activates the entire cerebrum bilaterally so rapidly that it produces a generalized tonic-clonic seizure before a focal sign appears.

Most patients with epilepsy become neurologically normal between seizures, although overuse of anticonvulsants can dull alertness. Progressive mental deterioration is usually related to the neurologic disease that caused the seizures. Left temporal lobe epilepsy is associated with verbal memory abnormalities; right temporal lobe epilepsy sometimes causes visual spatial memory abnormalities. The outlook is best when no brain lesion is demonstrable.

### Methods of Treatment

Modulating synaptogenesis through administering compounds that are agonists or antagonists of thrombospondin, including thrombospondin polypeptides and fragments thereof. is used to promote an improved outcome from ischemic cerebral injury, or other neuronal injury, by inducing synaptogenesis and cellular changes that promote functional improvement. The methods are also used to enhance synaptogenesis in patients suffering from neurodegenerative disorders, e.g. Alzheimer's disease, epilepsy, etc.

Patients can suffer neurological and functional deficits after stroke, CNS injury, and neurodegenerative disease. The findings of the present invention provide a means to enhance synapse formation and to improve function after CNS damage or degeneration. The induction of neural connections induced by promoting synaptogenesis will promote functional improvement after stroke, injury, aging and neurodegenerative disease. The amount of increased synaptogenesis may comprise at least a measurable increase relative to a control lacking such treatment, for example at least a 10% increase, at least a 20% increase, at least a 50% increase, or more.

The thrombospondin agonists and/or antagonists of the present invention are administered at a dosage that enhances synaptogenesis while minimizing any side-effects. It is contemplated that compositions will be obtained and used under the guidance of a physician for in vivo use. The dosage of the therapeutic formulation will vary widely, depending upon the nature of the disease, the frequency of administration, the manner of administration, the clearance of the agent from the host, and the like.

The effective amount of a therapeutic composition to be given to a particular patient will depend on a variety of factors, several of which will be different from patient to patient. Utilizing ordinary skill, the competent clinician will be able to optimize the dosage of a particular therapeutic or imaging composition in the course of routine clinical trials.

Therapeutic agents, e.g. agonists or antagonists can be incorporated into a variety of formulations for therapeutic administration by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the compounds can be achieved in various ways, including oral, buccal, rectal, parenteral, intraperitoneal, intradermal, transdermal, intrathecal, nasal, intracheal, etc., administration. The active agent may be systemic after administration or may be localized by the use of regional administration, intramural administration, or use of an implant that acts to retain the active dose at the site of implantation.

One strategy for drug delivery through the blood brain barrier (BBB) entails disruption of the BBB, either by osmotic means such as mannitol or leukotrienes, or biochemically by the use of vasoactive substances such as bradykinin. The potential for using BBB opening to target specific agents is also an option. A BBB disrupting agent can be co-administered with the therapeutic compositions of the invention when the compositions are administered by intravascular injection. Other strategies to go through the BBB may entail the use of endogenous transport systems, including carrier-mediated transporters such as glucose and amino acid carriers, receptor-mediated transcytosis for insulin or transferrin, and active efflux transporters such as p-glycoprotein. Active transport moieties may also be conjugated to the therapeutic or imaging compounds for use in the invention to facilitate transport across the epithelial wall of the blood vessel. Alternatively, drug delivery behind the BBB is by intrathecal delivery of therapeutics or imaging agents directly to the cranium, as through an Ommaya reservoir.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lines within a range of circulating concentrations that include the ED₅₀ with low toxicity. The dosage can vary within this range depending upon the dosage form employed and the route of administration utilized.

The pharmaceutical compositions described herein can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, intrathecal, and intracranial methods.

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

The compositions of the invention may be administered using any medically appropriate procedure, e.g. intravascular (intravenous, intraarterial, intracapillary) administration, injection into the cerebrospinal fluid, intracavity or direct injection in the brain.

Intrathecal administration maybe carried out through the use of an Ommaya reservoir, in accordance with known techniques. (F. Balis et al., Am J. Pediatr. Hematol. Oncol. 11, 74, 76 (1989).

Where the therapeutic agents are locally administered in the brain, one method for administration of the therapeutic compositions of the invention is by deposition into or near the site by any suitable technique, such as by direct injection (aided by stereotaxic positioning of an injection syringe, if necessary) or by placing the tip of an Ommaya reservoir into a cavity, or cyst, for administration. Alternatively, a convection-enhanced delivery catheter may be implanted directly into the site, into a natural or surgically created cyst, or into the normal brain mmass. Such convection-enhanced pharmaceutical composition delivery devices greatly improve the diffusion of the composition throughout the brain mass. The implanted catheters of these delivery devices utilize high-flow microinfusion (with flow rates in the range of about 0.5 to 15.01/minute), rather than diffusive flow, to deliver the therapeutic composition to the brain and/or tumor mass. Such devices are described in U.S. Pat. No. 5,720,720, incorporated fully herein by reference.

The effective amount of a therapeutic composition to be given to a particular patient will depend on a variety of factors, several of which will be different from patient to patient. A competent clinician will be able to determine an effective amount of a therapeutic agent to administer to a patient. Dosage of the agent will depend on the treatment, route of administration, the nature of the therapeutics, sensitivity of the patient to the therapeutics, etc. Utilizing LD₅₀ animal data, and other information, a clinician can determine the maximum safe dose for an individual, depending on the route of administration. Utilizing ordinary skill, the competent clinician will be able to optimize the dosage of a particular therapeutic composition in the course of routine clinical trials. The compositions can be administered to the subject in a series of more than one administration. For therapeutic compositions, regular periodic administration will sometimes be required, or may be desirable. Therapeutic regimens will vary with the agent, e.g. some agents may be taken for extended periods of time on a daily or semi-daily basis, while more selective agents may be administered for more defined time courses, e.g. one, two three or more days, one or more weeks, one or more months, etc., taken daily, semi-daily, semi-weekly, weekly, etc.

Formulations may be optimized for retention and stabilization in the brain. When the agent is administered into the cranial compartment, it is desirable for the agent to be retained in the compartment, and not to diffuse or otherwise cross the blood brain barrier. Stabilization techniques include cross-linking, multimerizing, or linking to groups such as polyethylene glycol, polyacrylamide, neutral protein carriers, etc. in order to achieve an increase in molecular weight.

Other strategies for increasing retention include the entrapment of the agent in a biodegradable or bioerodible implant. The rate of release of the therapeutically active agent is controlled by the rate of transport through the polymeric matrix, and the biodegradation of the implant. The transport of drug through the polymer barrier will also be affected by compound solubility, polymer hydrophilicity, extent of polymer cross-linking, expansion of the polymer upon water absorption so as to make the polymer barrier more permeable to the drug, geometry of the implant, and the like. The implants are of dimensions commensurate with the size and shape of the region selected as the site of implantation. Implants may be particles, sheets, patches, plaques, fibers, microcapsules and the like and may be of any size or shape compatible with the selected site of insertion.

The implants may be monolithic, i.e. having the active agent homogenously distributed through the polymeric matrix, or encapsulated, where a reservoir of active agent is encapsulated by the polymeric matrix. The selection of the polymeric composition to be employed will vary with the site of administration, the desired period of treatment, patient tolerance, the nature of the disease to be treated and the like. Characteristics of the polymers will include biodegradability at the site of implantation, compatibility with the agent of interest, ease of encapsulation, a half-life in the physiological environment.

Biodegradable polymeric compositions which may be employed may be organic esters or ethers, which when degraded result in physiologically acceptable degradation products, including the monomers. Anhydrides, amides, orthoesters or the like, by themselves or in combination with other monomers, may find use. The polymers will be condensation polymers. The polymers may be cross-linked or non-cross-linked. Of particular interest are polymers of hydroxyaliphatic carboxylic acids, either homo- or copolymers, and polysaccharides. Included among the polyesters of interest are polymers of D-lactic acid, L-lactic acid, racemic lactic acid, glycolic acid, polycaprolactone, and combinations thereof. By employing the L-lactate or D-lactate, a slowly biodegrading polymer is achieved, while degradation is substantially enhanced with the racemate. Copolymers of glycolic and lactic acid are of particular interest, where the rate of biodegradation is controlled by the ratio of glycolic to lactic acid. The most rapidly degraded copolymer has roughly equal amounts of glycolic and lactic acid, where either homopolymer is more resistant to degradation. The ratio of glycolic acid to lactic acid will also affect the brittleness of in the implant, where a more flexible implant is desirable for larger geometries. Among the polysaccharides of interest are calcium alginate, and functionalized celluloses, particularly carboxymethylcellulose esters characterized by being water insoluble, a molecular weight of about 5 kD to 500 kD, etc. Biodegradable hydrogels may also be employed in the implants of the subject invention. Hydrogels are typically a copolymer material, characterized by the ability to imbibe a liquid. Exemplary biodegradable hydrogels which may be employed are described in Heller in: Hydrogels in Medicine and Pharmacy, N. A. Peppes ed., Vol. III, CRC Press, Boca Raton, Fla., 1987, pp 137-149.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration.

### Gene Delivery

One approach for modulating synaptogenesis involves gene therapy. In such methods, sequences encoding thrombospondin or fragments thereof are introduced into the central nervous system, and expressed, as a means of providing thrombospondin activity to the targeted cells. To genetically modify neurons that are protected by the BBB, two general categories of approaches have been used. In one type of approach, cells are genetically altered, outside the body, and then transplanted somewhere in the CNS, usually in an area inside the BBB. In the other type of approach, genetic “vectors” are injected directly into one or more regions in the CNS, to genetically alter cells that are normally protected by the BBB. It should be noted that the terms “transfect” and “transform” are used interchangeably herein. Both terms refer to a process which introduces a foreign gene (also called an “exogenous” gene) into one or more preexisting cells, in a manner which causes the foreign gene(s) to be expressed to form corresponding polypeptides.

A preferred approach aims to introduce into the CNS a source of a desirable polypeptide, by genetically engineering cells within the CNS. This has been achieved by directly injecting a genetic vector into the CNS, to introduce foreign genes into CNS neurons “in situ” (i.e., neurons which remain in their normal position, inside a patient's brain or spinal cord, throughout the entire genetic transfection or transformation procedure).

Useful vectors include viral vectors, which make use of the lipid envelope or surface shell (also known as the capsid) of a virus. These vectors emulate and use a virus's natural ability to (i) bind to one or more particular surface proteins on certain types of cells, and then (ii) inject the virus's DNA or RNA into the cell. In this manner, viral vectors can deliver and transport a genetically engineered strand of DNA or RNA through the outer membranes of target cells, and into the cells cytoplasm. Gene transfers into CNS neurons have been reported using such vectors derived from herpes simplex viruses (e.g., European Patent 453242, Breakfield et al 1996), adenoviruses (La Salle et al 1993), and adeno-associated viruses (Kaplitt et al 1997).

Non-viral vectors typically contain the transcriptional regulatory elements necessary for expression of the desired gene, and may include an origin of replication, selectable markers and the like, as known in the art. The non-viral genetic vector is then created by adding, to a gene expression construct, selected agents that can aid entry of the gene construct into target cells. Several commonly-used agents include cationic lipids, positively charged molecules such as polylysine or polyethylenimine, and/or ligands that bind to receptors expressed on the surface of the target cell. For the purpose of this discussion, the DNA-adenovirus conjugates described by Curiel (1997) are regarded as non-viral vectors, because the adenovirus capsid protein is added to the gene expression construct to aid the efficient entry of the gene expression construct into the target cell.

In cationic gene vectors, DNA strands are negatively charged, and cell surfaces are also negatively charged. Therefore, a positively-charged agent can help draw them together, and facilitate the entry of the DNA into a target cell. Examples of positively-charged transfection agents include polylysine, polyethylenimine (PEI), and various cationic lipids. The basic procedures for preparing genetic vectors using cationic agents are similar. A solution of the cationic agent (polylysine, PEI, or a cationic lipid preparation) is added to an aqueous solution containing DNA (negatively charged) in an appropriate ratio. The positive and negatively charged components will attract each other, associate, condense, and form molecular complexes. If prepared in the appropriate ratio, the resulting complexes will have some positive charge, which will aid attachment and entry into the negatively charged surface of the target cell. The use of liposomes to deliver foreign genes into sensory neurons is described in various articles such as Sahenk et al 1993. The use of PEI, polylysine, and other cationic agents is described in articles such as Li et al 2000 and Nabel et al 1997.

An alternative strategy for introducing DNA into target cells is to associate the DNA with a molecule that normally enters the cell. This approach was demonstrated in liver cells in U.S. Pat. No. 5,166,320 (Wu et al 1992). An advantage of this approach is that DNA delivery can be targeted to a particular type of cell, by associating the DNA with a molecule that is selectively taken up by that type of target cell. A limited number of molecules are known to undergo receptor mediated endocytosis in neurons. Known agents that bind to neuronal receptors and trigger endocytosis, causing them to enter the neurons, include (i) the non-toxic fragment C of tetanus toxin (e.g., Knight et al 1999); (ii) various lectins derived from plants, such as barley lectin (Horowitz et al 1999) and wheat germ agglutinin lectin (Yoshihara et al 1999); and, (iii) certain neurotrophic factors (e.g., Barde et al 1991). At least some of these endocytotic agents undergo “retrograde” axonal transport within neuron. The term “retrograde”, in this context, means that these molecules are actively transported, by cellular processes, from the extremities (or “terminals”) of a neuron, along an axon or dendrite, toward and into the main body of the cell, where the nucleus is located. This direction of movement is called “retrograde”, because it runs in the opposite direction of the normal outward (“anterograde”) movement of most metabolites inside the cell (including proteins synthesized in the cell body, neurotransmitters synthesized by those proteins, etc.).

### Compound Screening

In one aspect of the invention, candidate agents are screened for the ability to modulate synaptogenesis, which agents may include candidate thrombospondin derivatives, variants, fragments, mimetics, agonists and antagonists. Such compound screening may be performed using an in vitro model, a genetically altered cell or animal, or purified protein. A wide variety of assays may be used for this purpose. In one embodiment, compounds that are predicted to be antagonists or agonists of thrombospondin are tested in an in vitro culture system, as described below.

For example, candidate agents may be identified by known pharmacology, by structure analysis, by rational drug design using computer based modeling, by binding assays, and the like. Various in vitro models may be used to determine whether a compound binds to, or otherwise affects thrombospondin activity. Such candidate compounds are used to contact neurons in an environment permissive for synaptogenesis. Such compounds may be further tested in an in vivo model for enhanced synaptogenesis.

Synaptogenesis is quantitated by administering the candidate agent to neurons in culture, and determining the presence of synapses in the absence or presence of the agent. In one embodiment of the invention, the neurons are a primary culture, e.g. of RGCs. Purified populations of RGCs are obtained by conventional methods, such as sequential immunopanning. The cells are cultured in suitable medium, which will usually comprise appropriate growth factors, e.g. CNTF; BDNF; etc. As a positive control, soluble thrombospondin, e.g. TSP1, TSP2, etc. may be added to certain wells. The neural cells, e.g. RCGs, are cultured for a period of time sufficient allow robust process outgrowth and then cultured with a candidate agent for a period of about 1 day to 1 week, to allow synapse formation. For synapse quantification, cultures are fixed, blocked and washed, then stained with antibodies specific synaptic proteins, e.g. synaptotagmin, etc. and visualized with an appropriate reagent, as known in the art. Analysis of the staining may be performed microscopically. In one embodiment, digital images of the fluorescence emission are with a camera and image capture software, adjusted to remove unused portions of the pixel value range and the used pixel values adjusted to utilize the entire pixel value range. Corresponding channel images may be merged to create a color (RGB) image containing the two single-channel images as individual color channels. Co-localized puncta can be identified using a rolling ball background subtraction algorithm to remove low-frequency background from each image channel. Number, mean area, mean minimum and maximum pixel intensities, and mean pixel intensities for all synaptotagmin, PSD-95, and colocalized puncta in the image are recorded and saved to disk for analysis.

The term “agent” as used herein describes any molecule, e.g. protein or pharmaceutical, with the capability of modulating synaptogenesis, particularly through a thrombospondin signaling pathway. Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Generally a plurality of assay mixtures are run in parallel with different agent concentrations to obtain a differential response to the various concentrations. Typically one of these concentrations serves as a negative control, i.e. at zero concentration or below the level of detection.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Test agents can be obtained from libraries, such as natural product libraries or combinatorial libraries, for example.

Libraries of candidate compounds can also be prepared by rational design. (See generally, Cho et al., Pac. Symp. Biocompat. 305-16, 1998); Sun et al., J. Comput. Aided Mol. Des. 12:597-604, 1998); each incorporated herein by reference in their entirety). For example, libraries of phosphatase inhibitors can be prepared by syntheses of combinatorial chemical libraries (see generally DeWitt et al., Proc. Nat. Acad. Sci. USA 90:6909-13, 1993; International Patent Publication WO 94/08051; Baum, Chem. & Eng. News, 72:20-25, 1994; Burbaum et al., Proc. Nat. Acad. Sci. USA 92:6027-31, 1995; Baldwin et al., J. Am. Chem. Soc. 117:5588-89, 1995; Nestler et al., J. Org. Chem. 59:4723-24, 1994; Borehardt et al., J. Am. Chem. Soc. 116:373-74, 1994; Ohlmeyer et al., Proc. Nat. Acad. Sci. USA 90:10922-26, all of which are incorporated by reference herein in their entirety.)

A “combinatorial library” is a collection of compounds in which the compounds comprising the collection are composed of one or more types of subunits. Methods of making combinatorial libraries are known in the art, and include the following: U.S. Pat. Nos. 5,958,792; 5,807,683; 6,004,617; 6,077,954; which are incorporated by reference herein. The subunits can be selected from natural or unnatural moieties. The compounds of the combinatorial library differ in one or more ways with respect to the number, order, type or types of modifications made to one or more of the subunits comprising the compounds. Alternatively, a combinatorial library may refer to a collection of “core molecules” which vary as to the number, type or position of R groups they contain and/or the identity of molecules composing the core molecule. The collection of compounds is generated in a systematic way. Any method of systematically generating a collection of compounds differing from each other in one or more of the ways set forth above is a combinatorial library.

A combinatorial library can be synthesized on a solid support from one or more solid phase-bound resin starting materials. The library can contain five (5) or more, preferably ten (10) or more, organic molecules that are different from each other. Each of the different molecules is present in a detectable amount. The actual amounts of each different molecule needed so that its presence can be determined can vary due to the actual procedures used and can change as the technologies for isolation, detection and analysis advance. When the molecules are present in substantially equal molar amounts, an amount of 100 picomoles or more can be detected. Preferred libraries comprise substantially equal molar amounts of each desired reaction product and do not include relatively large or small amounts of any given molecules so that the presence of such molecules dominates or is completely suppressed in any assay.

Combinatorial libraries are generally prepared by derivatizing a starting compound onto a solid-phase support (such as a bead). In general, the solid support has a commercially available resin attached, such as a Rink or Merrifield Resin. After attachment of the starting compound, substituents are attached to the starting compound. Substituents are added to the starting compound, and can be varied by providing a mixture of reactants comprising the substituents. Examples of suitable substituents include, but are not limited to, hydrocarbon substituents, e.g. aliphatic, alicyclic substituents, aromatic, aliphatic and alicyclic-substituted aromatic nuclei, and the like, as well as cyclic substituents; substituted hydrocarbon substituents, that is, those substituents containing nonhydrocarbon radicals which do not alter the predominantly hydrocarbon substituent (e.g., halo (especially chloro and fluoro), alkoxy, mercapto, alkylmercapto, nitro, nitroso, sulfoxy, and the like); and hetero substituents, that is, substituents which, while having predominantly hydrocarbyl character, contain other than carbon atoms. Suitable heteroatoms include, for example, sulfur, oxygen, nitrogen, and such substituents as pyridyl, furanyl, thiophenyl, imidazolyl, and the like. Heteroatoms, and typically no more than one, can be present for each carbon atom in the hydrocarbon-based substituents. Alternatively, there can be no such radicals or heteroatoms in the hydrocarbon-based substituent and, therefore, the substituent can be purely hydrocarbon.

Compounds that are initially identified by any screening methods can be further tested to validate the apparent activity. The basic format of such methods involves administering a lead compound identified during an initial screen to an animal that serves as a model for humans and then determining the effects on synaptogenesis. The animal models utilized in validation studies generally are mammals. Specific examples of suitable animals include, but are not limited to, primates, mice, and rats.

### EXPERIMENTAL

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### Example 1

The number of synapses between CNS neurons in culture is profoundly enhanced by a soluble signal secreted by astrocytes, which are identified herein as thrombospondins (TSPs), which are a necessary and sufficient component of the synapse-promoting activity of astrocyte-conditioned medium. TSPs induce ultrastructurally normal synapses that are presynaptically active but postsynaptically inactive. In vivo, TSPs are concentrated in astrocytes and at synapses throughout the developing brain, and mice deficient in both TSP1 and its ortholog TSP2 have a significant decrease in synapse number. These studies identify TSPs as the first known soluble synaptogenic protein in the CNS, and identify astrocytes as important contributors to synaptogenesis within the developing CNS.

TSPs are large oligomeric extracellular matrix proteins, about 500 kD, that mediate cell-cell and cell-matrix interactions by binding an array of membrane receptors, other extracellular matrix proteins, and cytokines. There are five TSPs, each encoded by a separate gene. Although several TSPs are expressed in the brain, the functions of these TSPs are unknown. TSP1 and TSP2 are closely related trimeric proteins that share the same set of structural and functional domains. TSP4, which is pentameric and has a different domain structure from TSP1 and TSP2, is present in the adult nervous system where it is localized to some CNS synapses as well as the neuromuscular junction.

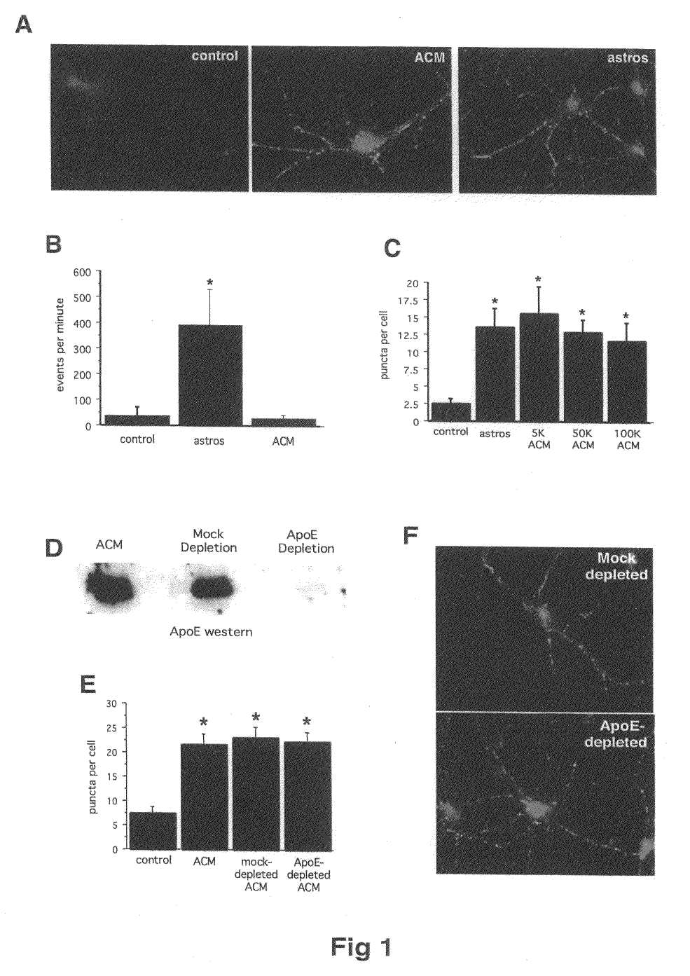

Astrocytes secrete at least two synaptogenic activities. In order to establish an assay for biochemical studies to identify synaptogenic activities secreted by astrocytes, we compared the ability of astrocyte-conditioned medium (ACM) and astrocyte feeding layers (“astros”) to induce synapses on RGCs (FIG. 1A). Synapses were detected as yellow puncta, representing colocalization of immunoreactivity to the pre- and postsynaptic markers synaptotagmin and PSD-95, respectively. Each yellow punctum corresponds to the site of a single functional synapse. As previously described, RGCs cultured for several days below a feeding layer of astrocytes have 7-fold more functional synapses than RGCs cultured alone, as assayed by whole-cell patch recording (FIG. 1B).

When RGCs were cultured in ACM there was an increase in the number of structural synapses (FIG. 1A), however, these synapses were not functional as indicated by the frequency of synaptic currents (FIG. 1B). Despite this lack of function, immunostaining showed that ACM induced as many structural synapses as an astrocyte feeding layer (FIG. 1C). This suggests that there are at least two signals secreted by astrocytes: one that is present in ACM that increases the number of structural synapses, and a second signal that induces functionality.

Apolipoprotein E particles do not contribute to the synaptogenic activity of astrocytes. Having established an assay in which ACM exhibits the same ability to induce synaptic puncta as a feeding layer, we next investigated the molecular weight of the synaptogenic ACM component. We found that all of the synapse-promoting activity in ACM was larger than 100 kD (FIG. 1C) and that the majority of the activity was still retained with a 300 kD cut-off filter.

To test whether ApoE-containing particles could be responsible for the activity, we immunodepleted ApoE-containing complexes from ACM (FIG. 1D). Despite depletion of virtually all of the ApoE protein, ACM induced the same number of synapses (FIG. 1E, F). Therefore, ApoE is not a required component of the synapse-promoting activity of ACM.

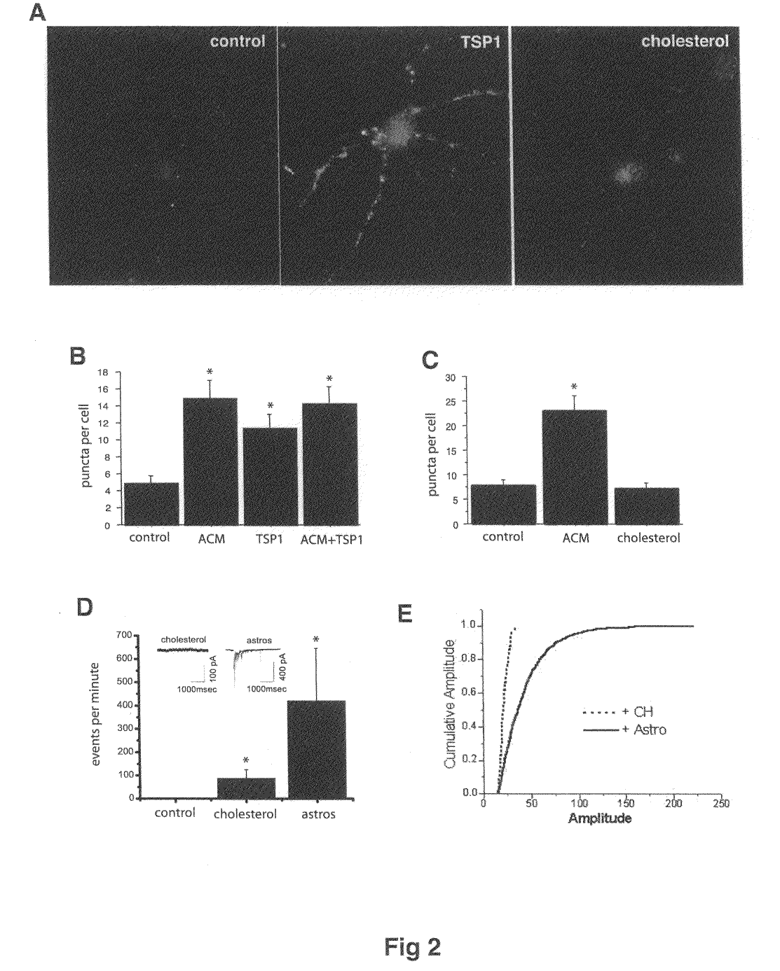

TSP1 is sufficient to mimic the ability of ACM to increase synapse number. The large size of the synaptogenic ACM activity, together with our observation that the activity is heparin binding, strongly suggested the possibility that the activity is an extracellular matrix protein. We next investigated the possibility that TSPs contribute to the synaptogenic activity of ACM because TSPs are made by astrocytes in vitro and in vivo, are well established as a promoters of cell adhesion in non-neural cells, and at least one family member is localized to synapses. First we directly tested whether TSP1 purified from human platelets has synaptogenic activity when added to RGCs in culture. TSP1 increased the number of synaptic puncta in RGCs to a similar degree as ACM (FIG. 2A, B). The number of puncta per RGC induced by TSP1 increased in a dose-dependent manner with concentrations of TSP1 ranging from 2 to 20 nM. This is the same nM concentration range that mediates known TSP1 functions outside the nervous system. In contrast to the results with TSP1, we found that treatment of RGCs with either ApoE or cholesterol (FIG. 2A, C) had no effect on synapse number.

Although TSP1 has been shown to enhance axon outgrowth when presented as a substrate, we found robust axon outgrowth occurred in RGCs in the absence of soluble TSP1, most likely due to the presence of laminin substrate and high levels of several neurotrophic factors in the medium. To confirm that TSP1 was not increasing synapse number secondarily to enhancing axon outgrowth, we measured the length of the longest neurite (the axon) on each RGC after one day of culture and found a statistically insignificance difference in axon length between control cultures and cultures plated with TSP1 (control: 196+15 μm, TSP1: 223+18 μm; means+SD, n=30). We also measured the total length of processes by dye filling individual neurons that had been cultured without or with TSP1. There was no difference in the access resistance or capacitance of the filled neurons without or with TSP1, indicating that the cells analyzed were the same size between the two groups and filled equally well. In addition, there was no increase in total process length of RGCs cultured with TSP1 (FIG. S1), confirming that the increase in synapse number is not due to a general increase in the length of axons or dendrites. In fact, total mean process length per RGC was reduced by TSP1, consistent with the possibility that cessation of outgrowth and synapse formation are linked processes.

To determine whether this synaptogenic effect was specific to TSP1, we tested a panel of extracellular matrix (ECM) molecules known to be secreted by astrocytes including fibronectin, vitronectin, tenascin C, osteonectin/SPARC, osteopontin, chondroitin sulfate proteoglycans (CSPGs) A and C, biglycan, and decorin, and various heparin sulfate proteoglycans (HSPGS) including agrin. None of these molecules had a significant effect on synapse number. We also tested a battery of peptide trophic factors and found they had no synaptogenic activity in this assay including CNTF, BDNF, insulin, TNF-α, II-6, GDNF, bFGF and TGβ.

Cholesterol enhances presynaptic efficacy but does not increase synapse number. Although we observed no effect of cholesterol on synapse number, we investigated whether cholesterol might be the astrocyte-secreted signal needed for synaptic function, since a previous report indicated that it strongly increased synaptic activity. When we used high-density bulk RGC cultures rather than low-density autaptic neurons, cholesterol only weakly increased the frequency of spontaneous synaptic events (FIG. 2D) and had no effect on cumulative current amplitude (FIG. 2E), a measure of postsynaptic sensitivity. In contrast, in autaptic cultures of RGCs cholesterol significantly increased quantal content (Fig. S2), a measure of presynaptic efficacy, but had no effect on mini-EPSC amplitudes (data not shown) as previously reported (8).

Thus the effect of cholesterol on synaptic activity appears primarily due to an increase in presynaptic efficacy, but its effects were much larger in autaptic cultures. It is possible that autaptic neurons are cholesterol-starved, since any cholesterol-containing particles they secrete are likely to be diluted into the medium and not readily available to other neurons due to the low culture density. Since cholesterol is a required component of synaptic vesicles, the increase in synaptic frequency in the absence of an increased number of synapses is likely explained either by a cholesterol-induced increase in vesicle number per synapse, resulting in an increase in release probability at the small number of existing synapses in RGCs cultured in the absence of astrocytes, or an increase in vesicles throughout the axon resulting in spontaneous and evoked neurotransmitter release that is primarily extrasynaptic. Regardless of mechanism, cholesterol treatment does not lead to a significant increase in synapse number or synaptic activity in bulk cultures, and thus cannot account for the large increase in synapse number and activity induced by astrocytes.

ACM and TSP1-induced synapses are ultrastructurally normal. Our previous studies showed that synapses induced by a feeding layer of astrocytes are ultrastructurally normal.

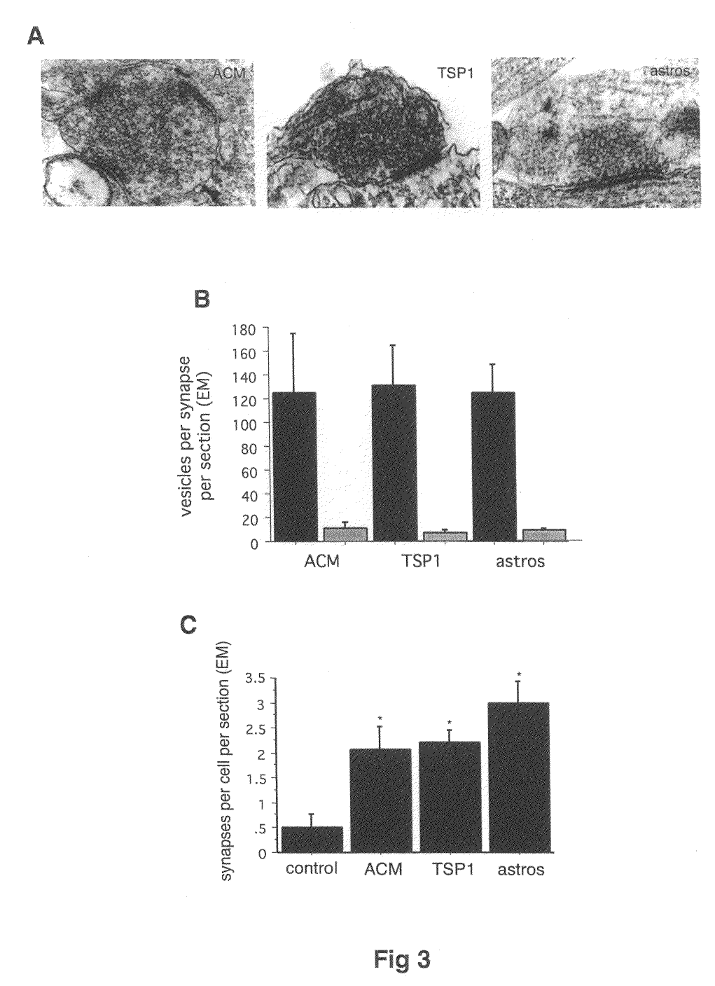

We used electron microscopy to study ACM- and TSP1-induced synapses in fine detail. Synapses induced by TSP1 and ACM were ultrastructurally identical to those induced by a feeding layer of astrocytes, which are electrophysiologically active. Pre- and postsynaptic specializations could be easily detected in RGCs cultured under both conditions as well as with an astrocyte-feeding layer (FIG. 3A). The number of vesicles per synapse and the number of docked vesicles per synapse were not statistically different between these three conditions, indicated by the finding that the numbers of vesicles per EM section were indistinguishable from one another (FIG. 3B). In agreement with our immunostaining data, using EM we found a comparable number of synapses were present in RGCs cultured with ACM, TSP1 or an astrocyte feeding layer, while the number in control cultures was much lower (FIG. 3C). These findings demonstrate that TSP1 is sufficient to induce ultrastructurally normal synapses, and provide evidence that the synaptic structures we detect by immunostaining likely correspond to the fully developed synaptic structures we observe by EM.

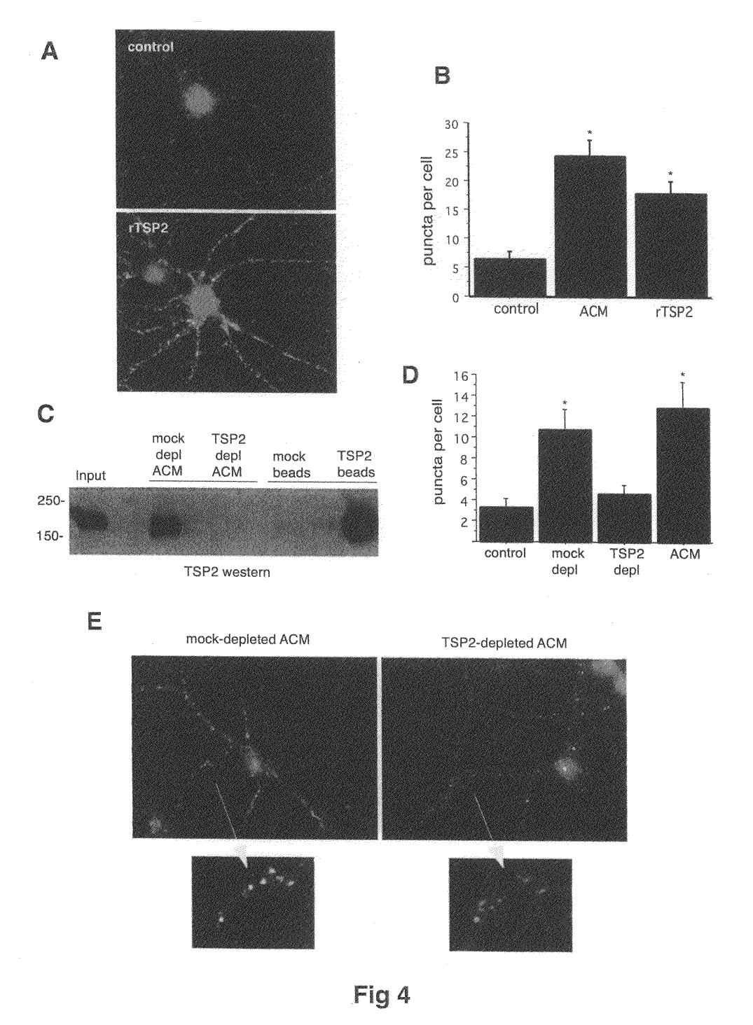

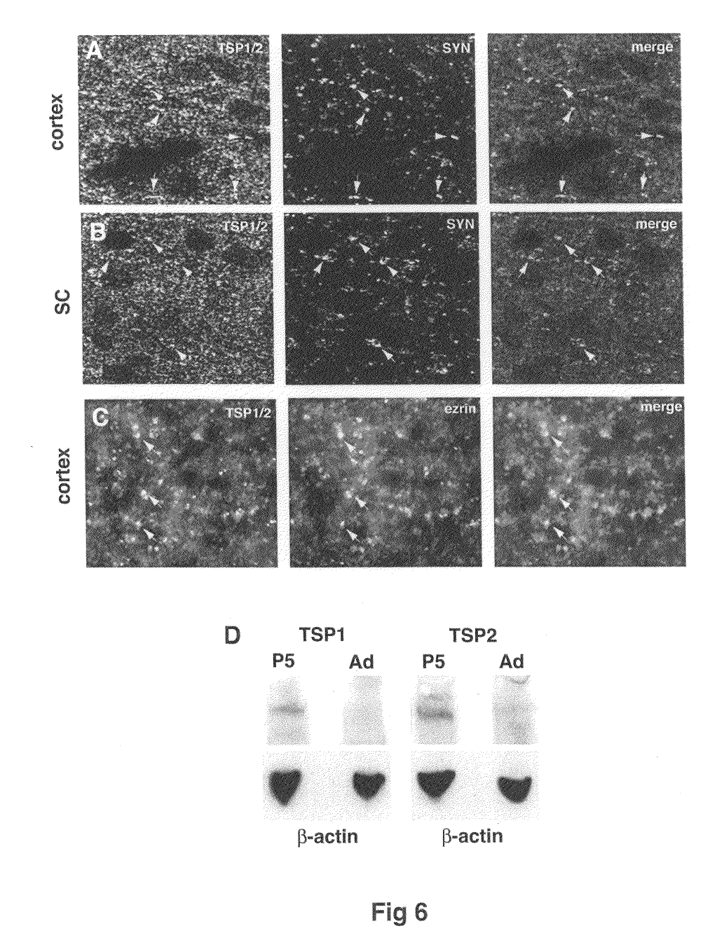

TSP2 is a necessary component of the synapse-promoting activity of ACM. We next investigated which TSPs are expressed by cultured astrocytes. Of the five members of the TSP family, TSP1 and TSP2 are highly related trimers and share common functional domains, while TSP3, TSP4 and COMP/TSP5 are pentameric and lack the procollagen and properdin domains present in TSP1 and TSP2. RT-PCR analysis of mRNA isolated from astrocytes in culture indicated expression of both TSP1 and TSP2. However, we were only able to detect protein for TSP2 by Western blotting of ACM and astrocyte cell lysate with TSP1- and TSP2-specific antibodies (FIG. 4C), even though the TSP1 antibody recognizes rat TSP1 in serum and rat brain lysate (FIG. 6).

Given that astrocytes secrete TSP2 protein, we asked whether TSP2 is synaptogenic. We found that recombinant TSP2 (rTSP2) increased synapse number in RGCs to a similar degree as TSP1 (FIG. 4. A, B). This finding indicates that TSP1 and TSP2 share a common domain(s) that is functional in synaptogenesis. In addition, the fact that synaptogenic activity is retained in the recombinant protein provides evidence that the activity of purified platelet TSP1 is not due to co-purification of a TSP-binding platelet protein. This conclusion is further supported by the lack of visible contaminating proteins in the recombinant TSP2 used for these experiments when analyzed by Coomassie staining.

Is TSP2 a necessary component of the ACM activity? When we treated RGCs with ACM and TSP1 together, the increase in synapse number was not larger than that observed with treatment by either alone (FIG. 2B), suggesting that ACM and TSP1 share a common pathway. To test directly for a requirement for TSP2, we immunodepleted TSP2 from ACM with a TSP2-specific antibody (FIG. 4C). RGCs cultured for several days in TSP2-depleted ACM developed several-fold fewer synaptic puncta compared to non-depleted ACM, reducing the number of synapses induced to control levels (FIG. 4D). Interestingly, despite the lack of double-labeled synaptic puncta in RGCs cultured with TSP2-depleted ACM, there was still a significant increase in the number of single-labeled puncta containing non-overlapping synaptotagmin or PSD-95 immunoreactivity (FIG. 4E). These findings demonstrate that TSP2 is necessary for astrocytes to enhance synaptogenesis and suggest that TSP2 may normally enhance synaptogenesis by inducing or maintaining the alignment and/or adherence of pre- and postsynaptic specializations.

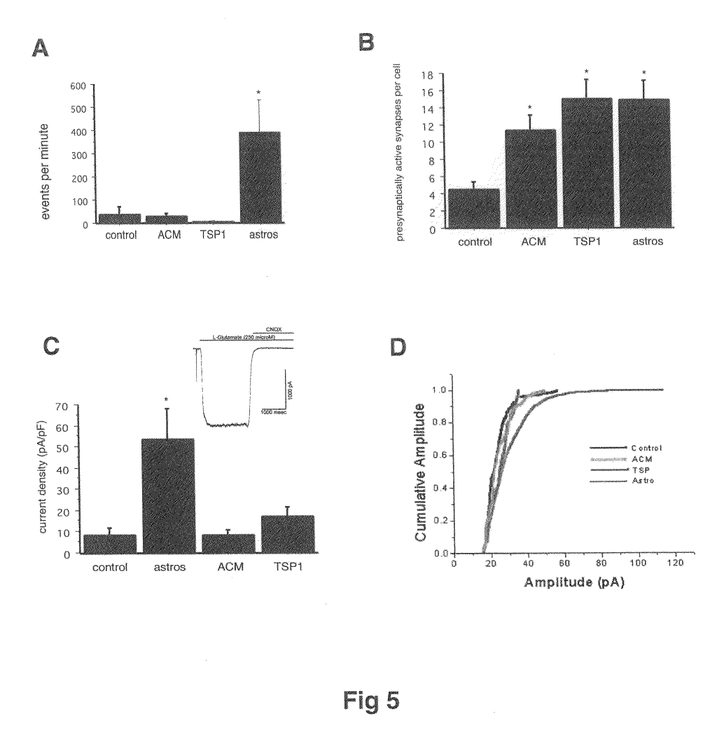

ACM and TSP1 induce formation of postsynaptically silent synapses. Despite their potent effects on increasing synaptic number neither ACM, TSP1 nor TSP2 greatly increased synaptic activity. Structurally normal synapses can be non-functional or “silent” either due to presynaptic mechanisms such as low probability of neurotransmitter release or postsynaptic mechanisms such as a lack of functional postsynaptic receptors. We used whole-cell patch clamp recording to determine the frequency and amplitude of synaptic events in RGCs cultured under various conditions. Astrocytes significantly increased the frequency of synaptic events while control, TSP1, ACM (FIG. 5A) and TSP2 (Fig. S3) did not.

We investigated whether this lack of function in TSP1- and ACM-induced synapses was due to a lack of presynaptic function, postsynaptic function, or both. To assess presynaptic function, we measured vesicular release using an antibody to the luminal domain of the vesicular protein synaptotagmin. When this antibody is added to live cells, it can only bind to its epitope when synaptic vesicles fuse with the presynaptic membrane and expose their luminal domain to the extracellular space. Vesicle membrane recycling through endocytosis leads to uptake of antibody that can then be visualized by immunoflourescence. We verified that the observed vesicular recycling was synaptic by double labeling with the postsynaptic marker PSD-95. Using this assay, we found that TSP1, ACM and an astrocyte feeding layer all increase the amount of spontaneous synaptic vesicular recycling in RGCs to a similar extent (FIG. 5B). Although the level of presynaptic activity induced by ACM is somewhat lower than that induced by purified TSP1, the difference was not statistically significant. In addition, the numbers of presynaptically active puncta per cell in all three conditions were similar to the numbers of structural synaptic puncta detected by immunostaining and EM. These results indicate that the majority of synapses induced by astrocyte feeding layers, ACM, and TSP1 are presynaptically active.

Synapses formed by RGCs in vitro and in vivo are largely non-NMDA receptor containing, primarily consisting of AMPA and kainate receptors with only a very small extrasynaptic NMDA receptor component. In order to assess postsynaptic function, we first examined postsynaptic responses of RGCs cultured in the presence of TSP1 or ACM to applied glutamate, and found that responses were not increased above control levels (FIG. 5C), indicating that there are either fewer glutamate receptors or fewer functional receptors expressed under these to conditions. To specifically assess synaptic receptor function we next measured the amplitudes of spontaneous miniature events (mEPSCs), the amount of postsynaptic current induced in response to the stochastic release of a single vesicle of glutamate. The cumulative mEPSC amplitude distribution shows that the synaptic events induced in RGCs cultured with either TSP1 or ACM are smaller than those induced by a feeding layer of astrocytes (FIG. 5D). By these measures, TSP1- and ACM-induced synapses are about 5-fold less sensitive to glutamate than astrocyte feeding layer-induced synapses. This difference could be accounted for either by a lack of glutamate receptors at the synapse or by the presence of non-functional receptors, and suggests that the second signal generated with an astrocyte feeding layer functions by either recruiting glutamate receptors to the synapse or by activating them.

Thus, whereas astrocyte-induced synapses are functional, both ACM and TSP1 induce structural synapses that are presynaptically active and postsynaptically silent. Importantly, this is not due to TSP1 inhibition of synaptic function, since TSP1 added to RGCs cultured with a feeding layer of astrocytes does not inhibit synaptic activity. The similar properties of the ACM- and TSP1-induced synapses provide further evidence that TSPs are a critical component of the synaptogenic activity of ACM. TSPs colocalize with synaptic markers and are expressed by astrocytes in vivo. We performed immunostaining with antibodies raised against TSP1 in postnatal brain, the age at which the bulk of synaptogenesis occurs. It is not clear whether these antibodies also recognize the highly related ortholog TSP2, so we refer to the immunoreactivity as TSP1/2. TSP1/2 immunoreactivity was observed widely in astrocytes throughout the postnatal cortex, superior colliculus, and retina, colocalizing with the synaptic marker synaptotagmin in both postnatal day 8 cortex (FIG. 6A) and superior colliculus (FIG. 6B). TSP1/2 immunoreactivity was not solely confined to synaptic regions; we also found extensive colocalization of TSP1/2 with ezrin, a marker of the fine astrocyte processes that ensheathe synapses in the postnatal CNS (22; FIG. 6C). Interestingly, TSP1/2 immunoreactivity largely disappeared in these brain regions by postnatal day 21, suggesting that trimeric TSPs may serve a transient function and are not required for maintenance of synapses.

In order to determine whether TSP1 and TSP2 proteins are present in the postnatal brain, we used other TSP1- and TSP2-specific antibodies, which work well for Western blotting but not immunostaining, to look at protein expression. Both TSP1 and TSP2 proteins were detected in extracts prepared from rat P5 cortex (FIG. 6D) and whole brain. As we observed for immunoreactivity, however, both TSP1 and TSP2 protein levels were very low or absent in adult brain. To further examine which TSPs are present in astrocytes in postnatal brain, we next performed RT-PCR on mRNA isolated from highly purified, acutely isolated astrocytes from P5 rat cortex. Both TSP1 and TSP2 mRNAs were detected. Taken together, these results show that both TSP1 and TSP2 are present in the developing brain, where they are highly localized to astrocytes, but are down regulated in adult brain.

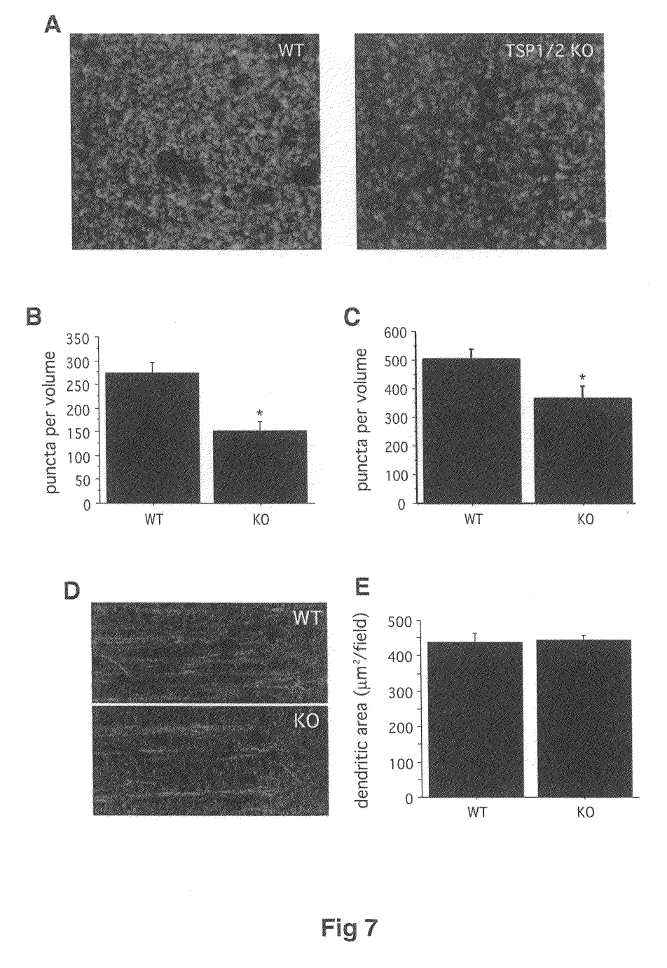

Role of TSP1 and TSP2 in CNS synaptogenesis in vivo. To determine if TSP1 and TSP2 play a role in CNS synapse formation in vivo, we quantified synapse number in brain cryosections prepared from wild type (WT) mice and mice lacking TSP1, TSP2, or both (TSP1/2 double-nulls; 23) by immunostaining with antibodies to the synaptic marker SV2 followed by confocal imaging. No decrease in synapse number was detected in TSP1 or TSP2 deficient mice. However, in the TSP1/2 double-null cerebral cortex there was a 40% decrease in synapse number at P8 and even by P21, a time when synapse number has normally plateaued, there was still a 25% decrease in synapse number compared to WT controls (FIG. 7 A-C). A similar decrease in synapse number was observed throughout TSP1/2 double-null brain sections including the superior colliculus. There was substantial variability between brain regions and mice, with decreases in cortical synapse number that ranged as high as 50% in some mice. Similar results were obtained using antibodies to other synaptic proteins including Bassoon and PSD-95.

To determine whether the effect of TSP1/2 deficiency on synapse number was direct and not secondary to effects on cell survival, proliferation or migration, we next counted the number of DAPI-stained nuclei per section in P21 cortex. We found no significant difference in the number of DAPI nuclei between WT and TSP1/2 double-null brains (85±10 nuclei per area WT; 97±10 nuclei per area TSP1/2 double-null, p=0.4). In addition, there was no obvious difference in the morphology of cortical structures or layers. To determine whether the effect of deleting TSP1/2 on synapse number was due to defects in dendritic arborization, we quantified the density of dendritic fields in synaptic areas of the cortex. We found no significant morphological difference in dendritic structures or dendritic arbor density between WT and TSP 1/2 double-null brains at P21 or P8 (FIG. 7 D,E). These data, together with the persistent decrease in synapse number at the nearly adult age of P21, provide evidence that the decrease in synapse number in TSP1/2-deficient mice cannot be explained by a decrease in cell number or dendritic number or length, but rather is due to a specific inability to form a normal number of synapses. These in vivo data, together with our in vitro data, show that TSPs play a crucial role in the promotion of CNS synaptogenesis in vitro and in vivo.

The results reported here support several conclusions. Our findings provide unequivocal evidence that soluble proteins can trigger synaptogenesis. We have identified the trimeric TSPs, TSP1, TSP2, TSP3, TSP4 and TSP5, as the first known soluble proteins that are sufficient to induce the formation of ultrastructurally normal CNS synapses. In contrast, cholesterol bound to ApoE is not synaptogenic but strongly enhances presynaptic efficacy. Unlike proteins such as NARP, ephrins, and agrin that preferentially stimulate postsynaptic differentiation, we found that TSP1 and TSP2 were sufficient to induce synaptic adhesions exhibiting both pre- and postsynaptic differentiation.

TSP2 is necessary for the ability of astrocytes to induce the formation of structural synapses between RGCs in vitro. TSP1 and TSP2 are both expressed in the postnatal but not adult CNS, where they are concentrated in astrocyte processes surrounding synapses. Finally, mice lacking both TSP1 and TSP2 have a substantially reduced number of synapses indicating that these TSPs help to promote normal CNS synaptogenesis in vivo. As we did not observe any obvious synapse loss in TSP1 or TSP2 single knockouts, but found a 25% decrease in the absence of both TSPs, it is quite possible that the degree of synapse loss might be substantially larger in the absence of additional TSP family members, as both TSP3 and TSP4 may be expressed in adult brain. Alternatively, TSPs may induce only specific synapse types.

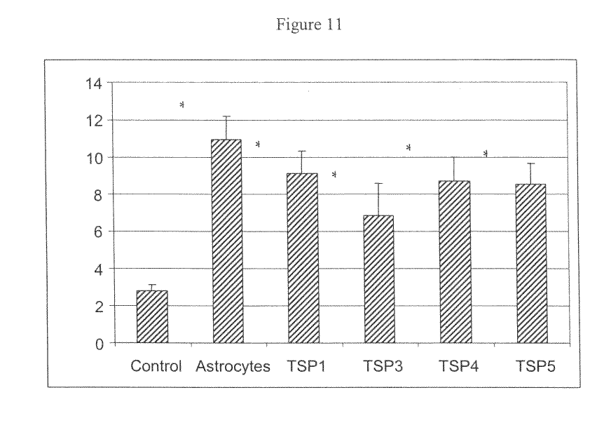

In addition, it is shown in FIG. 11 that TSP3, TSP4 and TSP 5 are equally active in promoting synaptogenesis. Taken together, these findings show that TSPs promote CNS synaptogenesis and strongly implicate astrocytes as active participants in CNS synaptogenesis in vivo.

The increase in synapse number by TSPs could be caused by an increase in formation of new synapses, stabilization of existing synapses, or both. Because RGC synapses are rapidly lost when astrocytes are removed, the simplest possibility is that TSPs act by stabilization. The well-known ability of TSPs to promote cell adhesion fits well with this possibility. In addition, RGCs cultured in TSP2-depleted ACM failed to form synapses but exhibited a large number of pre- and postsynaptic specializations that were not juxtaposed. This could be a result of initial synapse formation followed by destabilization, and is reminiscent of the misalignment phenotype that occurs at the neuromuscular junction in the absence of laminin α4, where synaptic specializations are present but not precisely apposed. A number of known TSP receptors that mediate the ability of TSPs to enhance adhesion in other tissues are concentrated at CNS synaptic locations, including the CD47 integrin-associated protein (CD47/IAP), a variety of integrins, and the low density lipoprotein receptor-related protein, LRP. Alternatively, TSPs are capable of functioning as de-adhesive proteins under certain circumstances and thus might switch growth cones from a neurite outgrowth mode into a synaptogenic mode by allowing them to de-adhere from outgrowth promoting substrates.

The identification of TSPs as the first known CNS synaptogenic proteins has important implications. Most importantly, our findings suggest that the levels of TSPs may control the timing of synaptogenesis as well as the number of synapses that the CNS is able to form. The effects of TSPs in promoting CNS synaptogenesis are likely to be instructive because we found that their effects are dose-dependent and their abundance in vivo is dynamically regulated during development, being low in late embryonic brain, higher in postnatal brain, and low or absent in the adult brain. The CNS levels of TSP1 and TSP2 correlate closely with the time interval when the rodent brain is able to form synapses during the first 3 postnatal weeks, a time period roughly concurrent with the critical period for synaptogenesis. The adult CNS is presently thought to have little ability to form new synapses.

As we have found that TSP1 and TSP2 are dramatically lower in adult brain, our results raise the important question of whether administration of exogenous TSP would restore the synaptogenic capacity of normal brain, or enhance the regeneration of new synapses in an injured CNS. Similarly, our findings have important implications for understanding the roles of astrocytes both in normal and injured brain. Release of astrocyte-derived TSPs could explain the close temporal and spatial correlation of astrocyte development with synapse development. Immature astrocytes in the postnatal brain express TSP1 and TSP2 mRNA, but then down regulate them in the mature brain. Remarkably, transplantation of immature astrocytes into an adult cat visual cortex is able to restore ocular dominance plasticity. Our findings indicate that astrocyte-derived TSPs contributed to this reemergence of synaptic plasticity in the adult brain.

Although TSP1 and TSP2 levels are normally low in the adult brain, reactive astrocytes and activated microglia express these proteins. Reemergence of TSPs could thus help to explain the formation of unwanted, extra synapses that result in epilepsy at astrocytic scars, as well as help to explain the tendency of axotomized axons to synaptically differentiate and fail to regenerate when they contact reactive astrocytes. Drugs that agonize or antagonize TSPs will help to promote synaptic plasticity and repair in many CNS diseases.

### Methods

Purification and culture of RGCs. RGCs were purified by sequential immunopanning to greater than 99.5% purity from P5 Sprague-Dawley rats (Simonsen Labs, Gilroy, Calif.), as previously described (Barres et. al. (1988) Neuron 9, 791). Approximately 30,000 RGCs were cultured per well in 24-well plates (Falcon) on glass (Assistant) or Aclar 22C (Allied Signal) coverslips coated with poly-D-lysine (10 μg/ml) followed by laminin (2 μg/ml). RGCs were cultured in 600 μl of serum-free medium, modified from Bottenstein and Sato (1979), containing Neurobasal (Gibco), bovine serum albumin, selenium, putrescine, triiodo-thyronine, transferrin, progesterone, pyruvate (1 mM), glutamine (2 mM), CNTF (10 ng/ml), BDNF (50 ng/ml), insulin (5 μg/ml), and forskolin (10 μM). Recombinant human BDNF and CNTF were generously provided by Regeneron Pharmaceuticals.

Purified human platelet TSP1 was from either Sigma or Haematologic Technologies with similar results. Recombinant TSP2 was purified from serum-free medium conditioned by baculovirus-infected insect cells expressing mouse TSP2. Since purified TSP1 is readily available, we used this as the source of TSP in our experiments unless otherwise stated TSPs were used at a concentration of 5 μg/ml unless otherwise specified. RCGs were cultured for 4 days to allow robust process outgrowth and then cultured with TSPs for an additional 6 days. TTX and Picrotoxin from RBI. All other reagents were obtained from Sigma.

Preparation of Astrocytes and Acm. Cortical Glia were Prepared as Described by McCarthy, J. de Vellis, J. Cell Biol. 85, 890 (1980). Briefly, postnatal dayl-2 cortices were papain-digested and plated in tissue culture flasks (Falcon) in a medium that does not allow neurons to survive (Dulbecco's Modified Eagle Medium, fetal bovine serum (10%), penicillin (100 U/ml), streptomycin (100 μg/ml), glutamine (2 mM) and Na-pyruvate (1 mM). After 4 days non-adherent cells were shaken off of the monolayer and cells were incubated another 2-4 days to allow monolayer to refill. Medium was replaced with fresh medium containing AraC (10 μM) and incubated for 48 hours. Astrocytes were trypsinized and plated onto 24-well inserts (Falcon, 1.0 μm) or 10 cm tissue culture dishes.

For preparation of ACM, confluent cultures of astrocytes in 10 cm dishes were washed 3× in PBS and fed with 10 mls RGC medium (without CNTF, BDNF or forskolin). ACM was harvested after 4-6 days of conditioning, filtered through a 0.2 μm syringe filter and concentrated 10× through a 5 KD molecular weight cut-off centrifuge concentrator (Millipore), unless otherwise indicated. ACM was used at a final concentration of 5× unless otherwise indicated. RCGs were cultured for 4 days to allow robust process outgrowth and then cultured with ACM or an astrocyte-feeding layer for an additional 6 days.

Electrophysiology. Membrane currents were recorded by whole-cell patch clamping at room temperature (18° to 22° C.) at a holding potential of −70 mV unless otherwise specified. Patch pipettes (3 to 10 megaOhms) were pulled from borosilicate capillary glass (WPI). For recordings of synaptic or whole cell glutamate currents, the bath solution contained (in mM) 120 NaCl, 3 CaCl2, 2MgCl2, 5KCl, and 10 Hepes (pH 7.3). The internal solution contained (in mM) 100 K-gluconate, 10 KCl, 10 EGTA (Ca2+-buffered to 10-6), and 10 Hepes (pH 7.3). For recordings of autaptic currents, the internal solution contained (in mM) 122.5 K-gluconate, 8 NaCl, 10 Hepes, 0.2 EGTA, 2 Mg-ATP, 0.3 Na-GTP, 20 K2-creatine phosphate, and phosphocreatine kinase (50 U/ml). Currents were recorded using pClamp software for Windows (Axon Instruments, Foster City, Calif.). Glutamate and CNQX (250 mM) were rapidly applied by a quartz microtube array (Superfusion System, ALA scientific instruments, New York). Mini excitatory post-synaptic currents (mEPSCs) were analyzed using Mini Analysis Program (SynaptoSoft, Decatur, Ga.) and plotted using SigmaPlot (SPSS, Chicago, Ill.) or Origin (Microcal, Northampton, Mass.).

Synaptic assays. For synapse quantification, cultures were fixed for. 7 min in 4% paraformaldehyde (PFA), washed 3× in phosphate buffered saline (PBS) and blocked in 100 μL of blocking buffer (50% Antibody Buffer (0.5% bovine serum albumin, 0.5% Triton X-100, mM NaPO4, 750 mM NaCl, 5% normal goat serum, and 0.4% NaN3, pH 7.4), 50% goat serum (NGS), 0.1% Triton-X) for 30 min. After blocking, cover slips were washed 3× in PBS and 100 μL of primary antibody solution was added to each cover slip, consisting of rabbit anti-synaptotagmin (cytosolic domain, Synaptic Systems) and mouse anti-PSD-95 (6G6-1C9 clone, Affinity Bio Reagents) diluted 1:500 in antibody buffer. Coverslips were incubated overnight at 4° C., washed 3× in PBS, and incubated with 100 μL of secondary antibody solution containing Alexa-594 conjugated goat anti-rabbit and Alexa-488 conjugated goat anti-mouse (Molecular Probes) diluted 1:1000 in antibody buffer. Following incubation for 2 h at room temperature, coverslips were washed five times in PBS and mounted in Vectashield mounting medium with DAPI (Vector Laboratories Inc) on glass slides (VWR Scientific). For presynaptic activity assay, rabbit synaptotagmin antiserum was generated by immunization with a peptide corresponding to the N-terminal luminal portion of synaptotagmin. This serum was added at 1:500 to live cultures and incubated for 6 hours. Cells were then washed 3× in DPSB, fixed and stained as above, except for the omission of synaptotagmin antibody from the primary antibody solution.

Mounted coverslips were imaged using Nikon Diaphot and Eclipse epifluorescence microscopes (Nikon). Healthy cells that were at least 2 cell diameters from their nearest neighbor were identified and selected at random by eye using DAPI fluorescence. 8-bit digital images of the fluorescence emission at both 594 nm and 488 nm were recorded for each selected cell using a cooled monochrome CCD camera and SPOT image capture software (Diagnostic Instruments, Inc). Each single-channel image was adjusted to remove unused portions of the pixel value range and the used pixel values were adjusted appropriately to utilize the entire pixel value range. Corresponding channel images were then merged to create a color (RGB) image containing the two single-channel images as individual color channels. These manipulations were performed automatically using the custom software package SpotRemover (©2001 Barry Wark).

Colocalized puncta were identified using a custom-written plug-in. Full documentation of the puncta-counting algorithm is available in the “Puncta Analyzer” plug-in's source code. Briefly, the rolling ball background subtraction algorithm was used to remove low-frequency background from each image channel. The puncta were “masked” in the single-channel image by thresholding the image so that only legitimate synaptic puncta remained above threshold. ImageJ's “Particle Analyzer” plug-in was then used to identify and characterize puncta within each channel. Puncta in different color channels were defined as colocalized if the centers of two circles, centered at the puncta's centroids and with areas equal to the puncta's area, were less than the larger of the two circle's radius apart. Number, mean area, mean minimum and maximum pixel intensities, and mean mean pixel intensities for all synaptotagmin, PSD-95, and colocalized puncta in the image were recorded and saved to disk for later analysis.

Dye filling of neurons. Whole cell voltage-clamped neurons were dye filled with Alexa 488 hydrazine (10 mM, Molecular Probes). Neurons were held at −70 mV for 10 min to allow movement of the dye into the neuron. Distal processes were well filled with this protocol. Access resistances and whole cell capacitance were measured and no difference was found between neurons cultured in the presence or absence of TSP (P>0.5), indicating that the access of the dye into the cell was the same in both conditions and that the size of the neurons was equivalent under both conditions. CCD images of individual cells were quantified using Metamorph (Universal Imaging Corporation).

Immunodepletion and Western analysis. 10×ACM was incubated with 20 μL goat anti-ApoE (a generous gift from Karl Weisgraber, UCSF) or 10 μL rabbit anti-TSP2 serum overnight at 4° C. Primary antibodies with bound proteins were removed from ACM by incubation with 20 μL Protein G or Protein A-Sepharose beads (Pierce), respectively, for 2 h at 4° C. followed by centrifugation to separate the supernatant, and a sample was saved for Western blotting before addition to RGC cultures. For preparation of rat cortical lysates P5 or adult rat cortices were homogenized in 20 volumes w/v lysis buffer [25 mM Tris 7.4, 150 mM NaCl, Complete Protease Inhibitor Cocktail (Roche)]. After homogenization, sodium deoxycholate was added to a final concentration of 1% and homogenate was solubilized at 4° C. for 30 min with rocking. Lysates were cleared by centrifugation at 16×g for 20 min at 4° C., and 30 μg of each lysate was used for Western analysis. Proteins in ACM or cortical lysates were resolved by SDS-PAGE and transferred onto PVDF (Millipore). Membranes were incubated in blocking buffer (PBS containing 0.1% Tween-20 and 5% nonfat milk) for 30 min at room temperature, followed by incubation for 1 hour or overnight at 4° C. in blocking buffer containing either rabbit anti-ApoE (1:500), mouse anti-TSP1 (1:250, BD Transduction), or mouse anti-TSP2 (1:250, BD Transduction). Immunoreactive proteins were detected using HRP-conjugated anti-rabbit or anti-mouse IgG (1:40,000; Jackson Immunoresearch) and visualized with a chemiluminescent substrate for HRP (SuperSignal West Pico; Pierce Chemicals).

Immunohistochemistry. Brain sections were dried 30 min at 37° C. followed by application of blocking buffer. Slides were washed 3×5 min in PBS. Primary used were diluted into antibody buffer as follows: TSP1 (P10, mouse monoclonal, Immunotech, 1:200 or Ab 8, Neomarkers, rabbit, 1:200), synaptotagmin (rabbit polyclonal, Synaptic Systems, 1:500), ezrin (monoclonal 3C12, Neomarkers, 1:200), SV2 (hybridoma supernatant, Developmental Studies Hybridoma Bank, 1:30), Bassoon (Stressgen, 1:400), PSD-95 (monoclonal 6G6-1C9, Affinity Bioreagents, 1:250) and incubated overnight at 4° C. followed by 3× washes in PBS. Secondary Alexa-conjugated antibodies (Molecular Probes) were added at 1:1000 for 2 hours at RT. Slides were washed 3× in PBS and mounted in Vectashield plus DAPI.

Electron microscopy. Cells were prepared for EM as previously described (Ullian et al., 2001). Briefly, cells were washed in 0.1 M phosphate buffer (pH 7.2) and fixed 30 min in 2% glutaraldehyde buffered with 0.1 M sodium phosphate (pH 7.2) at 4° C. After rinsing with buffer, specimens were stained en bloc with 2% aqueous uranyl acetate for 15 min, dehydrated in ethanol, and embedded in poly/bed812 for 24 hours. Fifty-nanometer sections were post-stained with uranyl acetate and lead citrate and viewed with a Philips Electronic Instruments CM-12 transmission electron microscope.

Confocal analysis of synapse number. Images of immunostained brains were collected on a Leica SPS SP2 AOBS confocal microscope. Optical sections were line-averaged and collected at 0.28 μM intervals. Gain, threshold, and black levels were individually adjusted per section to cover the same range of pixel values, or were set for the WT sections and kept constant for all sections. In both cases equivalent results were obtained for the relative number of synapses in WT or KO animals. Stacks of 20 optical sections were quantified for synapse number by projecting a series of 5 optical sections, a number empirically determined to optically section the entirety of most synaptic puncta, and counting the number of synapses in each projection volume. Synapses were automatically counted using the ImageJ puncta analyzer program and the accuracy of the counts confirmed by counting by hand. N=6 hemispheres for P8 WT and KO and N=10 hemispheres and for P21 WT and KO. On average 3 stacks per hemisphere were obtained yielding a total of 18 stacks (72 optical sections) for synaptic puncta analysis for P8 brains and a total of 30 stacks (120 optical sections) for synaptic puncta analysis for P21 brains.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims.

As used herein the singular forms “a”, “and”, and “the” include plural referents unless the context clearly dictates otherwise. All technical and scientific terms used herein have the same meaning as commonly understood to one of ordinary skill in the art to which this invention belongs unless clearly indicated otherwise.

What is claimed is:

1. A method of promoting or inhibiting synaptogenesis comprising the step of:
administering a therapeutic amount of a thrombospondin agonist or antagonist to a patient in need of synaptogenesis promotion or inhibition.

2. The method according to claim 1, wherein synaptogenesis is enhanced in said patient.

3. The method according to claim 2, wherein said patient has suffered synapse loss as a result of senescence.

4. The method according to claim 2, wherein said patient has suffered synapse loss as a result of Alzheimer's disease

5. The method according to claim 2, wherein said patient has suffered a CNS or spinal cord injury.

6. The method according to claim 5, further comprising administration of neural progenitors, or an neurogenesis enhancer.

7. The method according to claim 2, wherein said synaptogenesis is at a neuromuscular junction.

8. The method according to claim 1, wherein synaptogenesis is inhibited in said patient.

9. The method according to claim 8, wherein said patient suffers from epilepsy.

10. A composition for promoting or inhibiting synaptogenesis comprising:
an effective amount of a thrombospondin agonist or antagonist sufficient to promote of inhibit synaptogenesis; and
a pharmaceutically acceptable carrier.

11. A method of screening a candidate agent for activity in enhancing synaptogenesis, the method comprising:
contacting a neural cell culture with a candidate agent, wherein said agent is an antagonist or agonist of thrombospondin signaling;
quantitating the formation of synapses in culture.
